SEQUENCE LISTING (14 sequences)

SEQ ID NO: 1 - DNA, 7604 bp, Nannochloropsis oceanica W2J3B
cacacatgctttcatatgatcattacaagctcatcccacctcctcttcctcaaatcaccaactacagggcatttacttgtatgggtggcgtgttagcctatgcgtggggcgttggctttgtgctgccgtacctgtttatggtgggcacaggcgaggcggcagcgattgcggcgaaagacattccgtcgatagcggtggacgtgttgggcgacctggtgcttgtggtagggattttgatcggagctagtctgatgctattgtttaagagcattacactgggattagcgtcgtttttcatgatattggcggcggctttgctcgttgtcgggcgccttgcacttttgcataatctgctcagtgaaatcgtcctcgtaagtgcagccatcgcgcttggctgttcggtcttttactgcgcttaccgggaccaaaaaaaaaacagaggcattttaacgtattggtgacgtcggcgtttggagcactattgatggtatgcggctctggtagttttggggcgaagagcctcctcattcaaggtctaacagctctatgcaccttcgactacgccgccattggtcgttgcagtctttatggccaaagcccatgtcatccgttcttcgccctactcacctgggtactggttagcattgttgccgtggcgttgcagctatatttcggggcagacacggagctgaagcaaaagctgctgggcttcttaaattccgatcgtcacgtttacacctcggtgccggacggcaaagggcaatgtacggccaggctgggcccacctgcgatgggaaggcatcagcggcaagagagcattaccaacagggcggcgtctgaggatttgggagtgaatattttggatttgcggcattcattggacgcggaaggaggggctcaccctaccatggataaggaggaacggcagctgtgcgtaagtctgctgaatatgtttgatgagatgcaaggggtgttcgggtttcagacgcataatggtgttaatcaggttgagcatttggttttgctgctgaagaatcagaagcggtatcaagacccggcgtttcagaaattgattcctgctggaaaggggccattgacttataatgtggagacggcgacacctgtggatgtgctgcatgacaagatgttcaaaaactacaagcagtggtgcgagtccctcaaagtacaaccgcattttaataccatatggtcaatggttccgcgagaggggctcatgccgggagcggcgcccgtgggcgagaagtggtttgacaccgacgcggcgaagctgaaaatgcacaatttgctgttgctactcttgatctggggggaggcgggaaatatccgtcatatgcctgagtgtttagcgtggttatatcacacttcggcagcttgcctgcgggcatccacgcatcagacgctagagaatgtggaggaggagtatttcttggtcaatgccgtcacccctatctacaaagtaattgctgtggacatgcagaaaaagaaagacttggatcatcacgataagaagaactatgatgatttcaatgaatttttctggtcccgacagtgcttggactttacctggacccctgcggacatgccggctgtgcaggcggctcgaaccaagaatgcacggggtgaatttggtggcgaggacgaagagggaaagacaccaccgctttctttgatcggtgagggattgaagagggggccaaagacattcattgagaagcgatcctggctgatgatcatgctggcgtttaggcgtttaattgactttcatgtggtgacttttttcttgttggcgatgcagggattctggttgaatttgcaatgggatgacccgtattatttccaaatgatgtcggccgtgtttttgttgatgaattgtttggggatcgtgtggagtattttggaggtatggacgggcttgcaggcggaaacaaattcgtgcgcggcgttcaagacgcggagggaggcgaaacatggggtaatgctccggttgctggcgcgatttgtcttcctttttttccaggtgaaattttatggcctatctcttgtgggaggagggttggatctgaagccggcgcagcacttgagtgccaaaagtgtgcagttggagaactggtggatgtacgtatggatctcggtggcgctgcacactgtgtggtttatcgagtgtgtgttccagtgctggccgtatctctcaaccttagtgttcgaatgccgcaatcactacgttaaggccttgcttgatattgtgtttcctcaatcgcggaattacacggggaaacgcgtatatgagccctttaagaaatggttggtgtactccattttctggttctttgtcgtcagtgtcaagatcgctttctcctaccaatttgaagtcactcctttggccttgcctgctttagagctggcagatgatcagattaatttcttgaaccagaatgtatatttgacaattgtattgatagtcgtgcggtggttgccattcgtagccatctatatgctggacatgataatcatctattcgctggccgctgggttggtggggctagttgtgggtctgattgagaagctgggtcaagtgagggatttcgctggtatccgtgagaacttcatgcggacgcccgagagcttcttttctcggttgattttcaacacggacgatactcggagcaagcgcagtcggaaggcctcggatgtttcggatttggggatgtcccgccggtttacagcgagtagaaacgacctagtggctgcagcagctgatgcagaggagcggcagccgttgatggctgcgcttaatgcgggcatgcaaaattttgggactggagcttcggctactggtggtagtaatgatggtagggcgtcgactgaccatcaatcggtggaaaatgcggatgcgttcatggatgtgggtacgactaaatggcgtgcgttttcgacggcttggaataaggttgtgttgaatctgcggtccactgacatcatcaataatgatgagcgggacatgctattgttccatttctttacgggttttgccaaggatatttatttgccagtatttcagacggcgggctcggtggagagggccgcgcggttgtgcgcggagaagggaaaagagttccgcaccttggctgagaaaggaaaggagctccgtgccttggccgatcaggtcgagttgcagatgcagaacgatccaaaccaccaccacaatcagccgtacaggaaggccatcgataacaacaaggcagagatgattaagctggatacggcattgtgggaggagttgtcaaaggataggacgatgcatgaagcagtggcggagacgcttgagttgagcctagaattcttgatgcgcatgcttggggaggatcatgtatcggacgtgaataaggttaagctgacgatggagcgtctgcaggaaagcatgaagggggacgatgcggagaagggaagggcgggggggagaaaggtgatgattttatcggggataaagctggaggaagtggataaggctgtcggagcgttgggcaagatggtcacggcgctgaaaagtgggttgcctcgacgtgtcatcaacccgaaccgcgtcaagcctgcaaagcacacgccgagtgcgcgggagggtcgagggacggtaacggtgggatcggcaatgaagaaggtgcgtagccgcgggtttatgagcaacctctccctctcctcccagaacctcgtggaagtccgggagcaggcggagggccaggcttccgcgtctacgcccacggccagctcgcagcctttacatgagttggacagtttgcgggataaggtgcgggaggcactgagagggtttttgggtgcggtgaaaaagatgttagtgtctggaccgttgtttaaagatgtggcggaagcagtggacaagattttgactggacagtttttttggtgtgatgtgtatgcatccaactccctggatcagttggccaagcctgaggtgaaggaacttgtgcacaagatcctggcgaaactccaaggactcctcaccctgcatgtgggggatgcagagcccaagagtgcggaggcccgtcggcggttgaccttttttgtgaattctttgttcatggatgtgccgaaggcgccctctattgggaatatgttgtcatggacggtagtgacgcctttttattctgaggacgtgctctatagcagaaaggatttggatgcggcgaatgaggacggggtaaaaaccttactgtatctccagacgctgtataaagcggattggaaaaatttccaggagcggttgtcgttgcgggatgatagtccgatttggacggggaaggtgaaagaggagattcgattgtgggcatcgatgagggcacaaacactgtcaaggaccgtgcagggtatgatgtattatgaggacgccctgcatgtgttgagtcagctcgaccatgacgtaccaatcgttgacccggaggccaacacttccgaccaattgattcaaaggaagtttgggtatgttgttgcctgtcaggtgtatgggaagctgaagaaggagcaggatagtaaggctgatgacatcgacttccttctgcgcaaattccccaatttgcgggtggcgtacattgatgaaaagcaaagtaagagcggggagtcttacttttattctgtcttaatccgtgctgctgatgacaagaagactattgaggagatctacagagtgcgcctccctgggaaccctatcttgggggagggtaagccggaaaatcagaatcatgccatgatttttagtaggggggagcacgtgcaagcgattgatatgaaccaagagggttactttgaagatgcatttaagatgcggaactttttgcaagagttcgcggtgacggggactcctgacatgcctacaacaattttgggttttagggagcatattttcacgggtgctatctcatcactggctaattatatggcgctgcaggagtattcgtttgtaaccttgggccagcgggtattgaatcggccgttgcgcatgagattgcattatggtcatccggatttgtttgataagcttttctttattcagaacggagggattagcaaggcgtctaaggggatcaatctctccgaggacattttcgcgggctacaacaaccttcttcgaggggggtcggttgaatttaaggaatacgtacaagtgggcaaaggccgagatgttggcatgcagcagatctataagttcgaggccaagctctcccagggagcagctgagcagtctatatctcgcgatgtctctcggatgctgggccgcgtggattttttccggctgctttcctactattttggtgggataggccattacctttcttcagtgttgacagtcgcggcgatctggctgttggtttatttactgcttggcttggcgttattcgagcgtgagaagataggggatcggccaatggtgcctattggtaccctacaagtggcgcttgctggtgtgggtttgctacagacagccccgctcttttgtgccttactattggagaggggaatttgggctgcgctgacggagctggcgcaggtgtttattagtgggggaccattgtattttgtgtttcatattcgcacacgggattacttttacacgcaaaccattttggcgggtggtgcagcgtatagggcgacggggaggggtttcgtgacgcagcatgcttcttttgcggagacattccggttttttgcgttttcccacctttatttggggctggagatgattgcagccttgattttatttgcgtgtttcacggacgtagggcagtatgtgggtcggacgtggagtttatggtttgcggcgttggcgtttttgtacgcccctttttggtttaatccaatgagttttgagtgggaaagagtgagggaggacttggtgacttttgaggcttggatgcggacaacgggtggctcagcgtcgaactcgtgggaaacttggtggaaggaggagaataagtgggtaaaagagctgaaaaacgtctcggccaggctttatcttgttttgcggtcgtcgatttggttgatggtggcaacggggttgctgtataaacctatcgttgtggatggaaaaatggaccaattgcaatacctgctggagcacctctttgtgttgtttctgctgtttgcgacaagtaactacctggaagggagaagcaggagccgcaaccatcagggtgagtacgcgattatccgtggccttatgattatcctggctataattgcggttagttttttcgtcgtcacggcccagcacacggagacattcaaatttttagtggccctttactacattgccgcctggtgtgccacggtcatgtatgtctccaacagcaagaccgataaccttgtaaaagcctttcacaaagcacacgactggctcctggccacttgctgcttcgtccccataggcatctgcaccataattcagttccccgcctacatccaaacctggctcctctaccacaatgccctctctcaaggcgtcgtcatcggagatcttatccgctacgcgcagaatagtcgggaaaccaccaatatcattgatgaacgcgccgatgcctcctcccttgcgtcaggcttgcctactcctcgttcatccaccatctctttgatgtccggggccaccagagctacaacagctacctctgccgctactaccgtgggaacccttcagatctccccagaggaaaagaccaccgaacgcattgtcgaaattgagggcagcggtgggggcggatataacatactatcccctccgacgggtaccaagaaaaagaatggaaagaatggcacagcctcaaaagcagcgacggaattgccatggcaggcatcggttcaagatgcgcaggatccgtcggtggcagcgccgccgctgcccaatattaacactaacgcggggacggtggagtcgtttcagttccgacagccgaccaattttccgacgcgcgagtgaagggagaagggtgagagggaggaatggagggaggagggagctcgggcaaggcatggttatggatgcagattgatagcgccgccttacgtttgctaatgtttatgattaggggaagggcaccaaaatagacgagccagccccacctagcaagagaagagagtagccatagacaccgcagcaatagcagcagtaccgggacgcgcttccctatgttggatacaggtaagccctgcacgtgtgtcatgcataaaggatagcaagaacgaggccgggccactatttccagcagcagactccagaaaaggccattttgggatgtaacttcattttgtatcaagagtggaagggaaaggaaaagaagaagagagaggaaagggcgaaggacacagcagagatagtgagtgagataaagggtgtacccactttttgggatgtacgacatggtgaaagagggacatgacataaaaatagagaaaatagaaggcgccgcttccttagtgaattcggtgggaagaagatctttgggagtccttgggaggggaacaagagggaaaaaggagataacatcagagattccatgagagtaacagattcacggatgtgg

SEQ ID NO: 2 - protein, 2171 aa, Nannochloropsis oceanica W2J3B
MVCGSGSFGAKSLLIQGLTALCTFDYAAIGRCSLYGQSPCHPFFALLTWVLVSIVAVALQLYFGADTELKQKLLGFLNSDRHVYTSVPDGKGQCTARLGPPAMGRHQRQESITNRAASEDLGVNILDLRHSLDAEGGAHPTMDKEERQLCVSLLNMFDEMQGVFGFQTHNGVNQVEHLVLLLKNQKRYQDPAFQKLIPAGKGPLTYNVETATPVDVLHDKMFKNYKQWCESLKVQPHFNTIWSMVPREGLMPGAAPVGEKWFDTDAAKLKMHNLLLLLLIWGEAGNIRHMPECLAWLYHTSAACLRASTHQTLENVEEEYFLVNAVTPIYKVIAVDMQKKKDLDHHDKKNYDDFNEFFWSRQCLDFTWTPADMPAVQAARTKNARGEFGGEDEEGKTPPLSLIGEGLKRGPKTFIEKRSWLMIMLAFRRLIDFHVVTFFLLAMQGFWLNLQWDDPYYFQMMSAVFLLMNCLGIVWSILEVWTGLQAETNSCAAFKTRREAKHGVMLRLLARFVFLFFQVKFYGLSLVGGGLDLKPAQHLSAKSVQLENWWMYVWISVALHTVWFIECVFQCWPYLSTLVFECRNHYVKALLDIVFPQSRNYTGKRVYEPFKKWLVYSIFWFFVVSVKIAFSYQFEVTPLALPALELADDQINFLNQNVYLTIVLIVVRWLPFVAIYMLDMIIIYSLAAGLVGLVVGLIEKLGQVRDFAGIRENFMRTPESFFSRLIFNTDDTRSKRSRKASDVSDLGMSRRFTASRNDLVAAAADAEERQPLMAALNAGMQNFGTGASATGGSNDGRASTDHQSVENADAFMDVGTTKWRAFSTAWNKVVLNLRSTDIINNDERDMLLFHFFTGFAKDIYLPVFQTAGSVERAARLCAEKGKEFRTLAEKGKELRALADQVELQMQNDPNHHHNQPYRKAIDNNKAEMIKLDTALWEELSKDRTMHEAVAETLELSLEFLMRMLGEDHVSDVNKVKLTMERLQESMKGDDAEKGRAGGRKVMILSGIKLEEVDKAVGALGKMVTALKSGLPRRVINPNRVKPAKHTPSAREGRGTVTVGSAMKKVRSRGFMSNLSLSSQNLVEVREQAEGQASASTPTASSQPLHELDSLRDKVREALRGFLGAVKKMLVSGPLFKDVAEAVDKILTGQFFWCDVYASNSLDQLAKPEVKELVHKILAKLQGLLTLHVGDAEPKSAEARRRLTFFVNSLFMDVPKAPSIGNMLSWTVVTPFYSEDVLYSRKDLDAANEDGVKTLLYLQTLYKADWKNFQERLSLRDDSPIWTGKVKEEIRLWASMRAQTLSRTVQGMMYYEDALHVLSQLDHDVPIVDPEANTSDQLIQRKFGYVVACQVYGKLKKEQDSKADDIDFLLRKFPNLRVAYIDEKQSKSGESYFYSVLIRAADDKKTIEEIYRVRLPGNPILGEGKPENQNHAMIFSRGEHVQAIDMNQEGYFEDAFKMRNFLQEFAVTGTPDMPTTILGFREHIFTGAISSLANYMALQEYSFVTLGQRVLNRPLRMRLHYGHPDLFDKLFFIQNGGISKASKGINLSEDIFAGYNNLLRGGSVEFKEYVQVGKGRDVGMQQIYKFEAKLSQGAAEQSISRDVSRMLGRVDFFRLLSYYFGGIGHYLSSVLTVAAIWLLVYLLLGLALFEREKIGDRPMVPIGTLQVALAGVGLLQTAPLFCALLLERGIWAALTELAQVFISGGPLYFVFHIRTRDYFYTQTILAGGAAYRATGRGFVTQHASFAETFRFFAFSHLYLGLEMIAALILFACFTDVGQYVGRTWSLWFAALAFLYAPFWFNPMSFEWERVREDLVTFEAWMRTTGGSASNSWETWWKEENKWVKELKNVSARLYLVLRSSIWLMVATGLLYKPIVVDGKMDQLQYLLEHLFVLFLLFATSNYLEGRSRSRNHQGEYAIIRGLMIILAIIAVSFFVVTAQHTETFKFLVALYYIAAWCATVMYVSNSKTDNLVKAFHKAHDWLLATCCFVPIGICTIIQFPAYIQTWLLYHNALSQGVVIGDLIRYAQNSRETTNIIDERADASSLASGLPTPRSSTISLMSGATRATTATSAATTVGTLQISPEEKTTERIVEIEGSGGGGYNILSPPTGTKKKNGKNGTASKAATELPWQASVQDAQDPSVAAPPLPNINTNAGTVESFQFRQPTNFPTRE

SEQ ID NO: 3 - DNA, 6801 bp, Nannochloropsis gaditana IC164
atgggcggcatgctggcctatttctggtgcgtcagctggatggagcccaatcttttcatctcagacggcagcgccgctgctgcagcagacatcccgcagacagcactagacgtgttaagcggcatcgtgatcctggcgggcacggccactggggctggcttgatgctcctgtggagaagcatctcaatcggagtgatttctggcttcttgacgttggcgctgctgatgctgttggtgggaggcgcctcgttttctggggccgccttcacggggcccgtagtcgtgcttctcgcctgcctcgcgggggtgggagcactcttacttgcgtatcggggcaatcgtatgtccaaacaacgtatgaatgtgatcgtgacgtctgcattcgggtcgctggttctggcatgctcatatgatccatggggcgcgaggaatttcttgctcggagacctggccgctgtcagcgtcctcgactgggcggccatcggtcactgtagccttgagaggggaggctgccatccgcgcgtggccctaggcatgtggctcgtctcaacatttttcgcttgccttgttcaagtgtaccttggaggagaccccgagctccggcagcaagtcctggcccttctacggcgcgaccaccacccgtaccagtcgctgcccgacgcaccgacccggaggtcagacgcgtcgaagcaggaaccggcagcccttcccaagcacctccgccaagacatcaacaaattcaagctagcagagttgggggtcaatattttggatttgcggcatcccgtggatgctgaagaggacggtcgctccagcagcatggacgaagaagagagtaagctgagcgccactctcctgtgtatgtttgaggagatacaagacgtttttggttttcagacccacagtggcgtcaatcaggtggagcacctagtccttcttttgatgaaccagaaacgctacgaggatcctgcctaccgggagttgatgccggcagggaaaggacccttgagcgacgaggcggtcgatgccggtcctgtaaaaatcctacacgacaagttgttcaagaactacaaacgctggtgcgcctccttgaaggttgctccccatttcgacacgataccccactcggaaagccgcggcacctcggcaagttggaatggctctggcttgggctcgacgggagggaagtggttcgagagagaaggggataaggtgaaaatgcacaatctgctcttattcctgcttatctggggcgaagctggtaatcttcgacacatgcccgagtgcatagcgtggctataccacaccactgctgcttgttttaagggctccaccctccagaccatcgaggccgtggaggaggagtactttctcacccacgccgtcacgcccatttacgcggtggtggcggtggacatgaagaaaagcaggatggaccacgtgaataagaagaactacgacgatttcaacgagttcttctggtctcgtcagtgtctggcgtacacatggacgccggaggacatgccggccgtgcaggcggcgagggccaagagagcggcgggcgagcatgcgcgaccgggggggggcgagaccggtctgatcgcccgggcgctgaagggtagccccaagacattcatggagaagcggtcctggctcatgatcatgctggctttccggcgcctcatcgacttccacgtcgtcactttcttcatcctggccgtgcagggtttctggctgaatttgcagtgggacgatccttactactaccagctcatgtcctccgtgttcatgctcatgaactctctgggaatcttctgggctaccctcgagatatgggccaccatgcaggatatacagagtccttgccctccgttcgaggtccgagaagaggcaaaacacggcgtcatgctgcgtctcctgacccgcttcgtcttcttgcttttccaagccaggtacttcgggctttccttagaagctgatgggctcgatttacttcctgatgaacgtttaagtgacaaatcggtgcagctggaggcctggtggatgtacgtgtggatctctgtggcccttcactcagtctggatccttgactgcgtcttccaggcctgtccgcctctctcgacgcaagtctttgagacccgcaaccactacgtcaaggcgctgctcgacatcatcttccctcagatgcgaacctacaccggcaagcgtgtgcatgagcccttccacaagtggtgcttatatttcctgttctggtccgtcgtcatcacagccaagatttgtttttcgtaccaatttgaagtttctcccctggcgctgccggctctggaactggcggatgatcaggttaactacctcaataagaacctgtatttgacaattttattgatcatagggcggtggctgcccttcgtggccatctacttgttggacatgattattgtctattccttggtcgcaggcgtcgtgggtttcttggtgggtctgtacgagagactcggccaagtatgcgacttcgctgggattcgcgagcactttatgcgcacgcctgagagtttttactcccgtctcatctacaattctgaagatcgtcgaccgaaacacagtcgcaaggcttctcacgtctctgatctaggcatgtctcgccggttcacgtccagtaggaacaatctcctggcagcggcacaggatgatgacgagcgcaagcccttggtagctaccaatacaagtggtatgcagcggttgggaaacggaatcagaagcaattacaacgggacttccacgcaaccgcattatgagtggatgaactgtgatgaggcctttttggatattggcaccaccaagtggtacgcgttcgctaccgcatggaacaaaatagttgaaaatctgcgtgagacagacatcattagcaacgacgagcgggacatgctccttttctattttttcaagggtctgagcaagagtatctacctccccgtgttccaaaccgctggctatgtggagaaggcggcgcggctctgcgctgaaaagggtaaagaatttcgtgcactacctaacgataacgtccacgatggagatcagagcttgaaacaaaaaagagatgcgatcaaatcagacaagcagcgcgtggatcgggagcttagggagctgctgaacaaagatcgaacagcgtacgaggcggtagctgagacgctcgaattgacgctggacttcttgaggcggatgctgggacccaagcacgcgcaagacgtgctggcggcaaccttcaccttggagagctttcagggaagcaatcgggtcatgacggtagagagagcagtcgaagaagggaatggacagggtatgggtcttattttggagtccctcagacttgaaaacgtggagaaggcggtagaagcattaggcaaagctgtctcggcgctcaaaagcggccttccccgtcgggtcatcaatcccaagcgggttgaaccagtgaagatggcgatcccaccacgtgaaaggggcggaatggtgacggtggggtcctcgatgaggagagttaggagcaaaggtttcatgagcaacctgtccctgtcgtcacaggatctcgtcgcggtcggagagcaggctgcggagggtgctgtccatcagtccccggcgcagccgcaagtagagctagacagtctccgagacaagataagagattcacttcgaatcttcttgagcactgtcaaagggattattgtaccaggcgcgccaaactatctccttgctgatgtagcgacggcaataaccaatgtgctgaacggccccttcttctgggatgactattatgcatcggaagagcttgaccgcttggcggagtccgaggcaaagtcggcggtgatgcccgttctggccaagcttcaagggctcctgacgctgcatgtgggcgatgcggagcctaaaagtgcagaggctcgtcggcgccttagtttcttcgtaaactccctcttcatggatgtacccaaggcaccttctatatcggatatgatgtcttggacggtgatcaccccattctacagcgaggatgttttgtacaacaggaaggatctcgaggcggcgaatgaggacggcgtcaataccttgctgtatcttcaaacgctttacaagtcggactggaaaaattttcaggagcgcctcggtctgcgaaatgacagcactagttgggcgggcaaggccaaggaggagatacggctttgggcatcgatgcgtgcgcagactctgtcacgcacagtgcaaggcatgatgtactacgaggacgcgcttcatatgctgagtgtcctggaccgggacccttcactgatgccaaatgcggagtccaacagtgtacagcagcttattaaacgaaagtttgggtatgtggtagcgtgtcaggtttacgggaagttaaaaaaggagcaggacagcaaggcggatgacattgatttcctccttcgccgttttcccagtctgcgcgtcgcgtacatcgatgaacgtcagagcaagagtggcgagtcttcctttttctctgtcttaatccgcgccaatgatgccggcacgggcatcgaggagatataccgcgtgcgtctgccgggcaatcctgtccttggtgaaggaaaaccggaaaatcaaaatcacgcgatgatatttagtcgcggcgaacacgtacaagcaatcgacatgaatcaagaaggatacttcgaggacgcttacaagatgcgtaattttctgcaagagttcgcattgacagggtctcctgacatgccgacgacaattttgggtttccgtgagcacatttttaccggggcagtctcatctttagccaattatatggctcttcaggaatattcattcgtgactctcggtcaaagggtacttaatcgaccgctgcgcatgcgcttacactacgggcatccggatttatttgacaagcttttcttcatgcagaacggggggattagtaaagcttccaagggaataaatctctctgaagacatttttgcgggttacaacaacttgctccgtggaggttctgtagaatttaaagagtacgtccaagtgggaaaaggtcgcgacgttggcatgcaacagatatataaatttgaggccaaactttctcagggtgccgccgaacaatcgatttctcgcgatgtgtatcgcatggtcaatagagtcgactttttccgccttcttacctactacttcggtggcatcgggcattacctatcttctgtacttacagtcgcggctatctggctcctggtttatgtgctcttaagcttatccctcttccagcacgaaaaaattggggatcggccaatggtgccgatcggcaccttacagatagtgcttgctggcgtaggaatccttcaaacgatgcctcttttttgcgccttgctgcttgagcgcggtgtctgggcttccctcacagagttagcccaggtttttatcagcggtggccctctatactttgttttccatatccgcactcgagattactactattctcagacgattcttgccggcggtgccgcgtacaaggctacgggtcggggattcgtgactcagcacgcgtcattcgccgaaacattcagatattttgccgcaagccacctctacctagggctcgagatggtcgccgcgttggtcctattcgcctgttacacggatgccgggcaatatgtgggccgaacgtggagcctgtggttcgcggctgtggcattcttgtacgctccattttggttcaaccccatgagtttcgaatgggagcgcgtgcgagaggacgttgaaacttttgtctcgtggatgtgcaccactgggggctccacgaaaaattcctgggagtcatggtggaaagaggaaaacggatggatcaaagcgctgggacccacggctaaagcgtatctcgtcggtcgctcatgtatttggctggttgtggccgccggattgctgtataaacctttgtacttgaatcgcaagttcagcggattgaactatcttctgtttcatctaggcatcctcctgggactttggcagttctatcggttcctggacaggaggggcaggacgcggaatctcccattgccttattgctgcacgcggcccacgaacatcgttatagggatgggcatcgtcttcctggtggctctcatcatcatacattccgagacgatcaaatttttcgtggctctgtactacctcggggcgtggattacggtggtcctctcagttttagggtttagagagcaggctaagatcttccattggattcatgactgggtcttggctgtcgtcttgattatccccatctttctatgcactatacttcagtttcctcggcatattcaaacgtggctgctgtaccacaacgctctttcccaaggcgtggtaataagcgacttgattcgtcacgcgcaaaacagccgcgaaatgtccaatacggatgatgagcgcgcgcaggctccccgttcacatgccttggcatcagctttactgaatacaccttcatctgtgaacctcagatcagcttattcaccggcatcaggcggtcccatgcagatctctcctgaggagaaaacaagagagcgtcttgttggcagtggtggtggcaacgggtttgataccacatcgggcgcttcctgcaaacgagagtcattcaaaagcggacagacacgaccagatcattctcagtcgacgagtcagcgcccacaccaagatccatctccagtgtcgccggcagcctctgagcaatccccagaggtgtttcaattccgccaaccgaccaattttccaacacgggaataa

SEQ ID NO: 4 - protein, 2266 aa, Nannochloropsis gaditana IC164
MGGMLAYFWCVSWMEPNLFISDGSAAAAADIPQTALDVLSGIVILAGTATGAGLMLLWRSISIGVISGFLTLALLMLLVGGASFSGAAFTGPVVVLLACLAGVGALLLAYRGNRMSKQRMNVIVTSAFGSLVLACSYDPWGARNFLLGDLAAVSVLDWAAIGHCSLERGGCHPRVALGMWLVSTFFACLVQVYLGGDPELRQQVLALLRRDHHPYQSLPDAPTRRSDASKQEPAALPKHLRQDINKFKLAELGVNILDLRHPVDAEEDGRSSSMDEEESKLSATLLCMFEEIQDVFGFQTHSGVNQVEHLVLLLMNQKRYEDPAYRELMPAGKGPLSDEAVDAGPVKILHDKLFKNYKRWCASLKVAPHFDTIPHSESRGTSASWNGSGLGSTGGKWFEREGDKVKMHNLLLFLLIWGEAGNLRHMPECIAWLYHTTAACFKGSTLQTIEAVEEEYFLTHAVTPIYAVVAVDMKKSRMDHVNKKNYDDFNEFFWSRQCLAYTWTPEDMPAVQAARAKRAAGEHARPGGGETGLIARALKGSPKTFMEKRSWLMIMLAFRRLIDFHVVTFFILAVQGFWLNLQWDDPYYYQLMSSVFMLMNSLGIFWATLEIWATMQDIQSPCPPFEVREEAKHGVMLRLLTRFVFLLFQARYFGLSLEADGLDLLPDERLSDKSVQLEAWWMYVWISVALHSVWILDCVFQACPPLSTQVFETRNHYVKALLDIIFPQMRTYTGKRVHEPFHKWCLYFLFWSVVITAKICFSYQFEVSPLALPALELADDQVNYLNKNLYLTILLIIGRWLPFVAIYLLDMIIVYSLVAGVVGFLVGLYERLGQVCDFAGIREHFMRTPESFYSRLIYNSEDRRPKHSRKASHVSDLGMSRRFTSSRNNLLAAAQDDDERKPLVATNTSGMQRLGNGIRSNYNGTSTQPHYEWMNCDEAFLDIGTTKWYAFATAWNKIVENLRETDIISNDERDMLLFYFFKGLSKSIYLPVFQTAGYVEKAARLCAEKGKEFRALPNDNVHDGDQSLKQKRDAIKSDKQRVDRELRELLNKDRTAYEAVAETLELTLDFLRRMLGPKHAQDVLAATFTLESFQGSNRVMTVERAVEEGNGQGMGLILESLRLENVEKAVEALGKAVSALKSGLPRRVINPKRVEPVKMAIPPRERGGMVTVGSSMRRVRSKGFMSNLSLSSQDLVAVGEQAAEGAVHQSPAQPQVELDSLRDKIRDSLRIFLSTVKGIIVPGAPNYLLADVATAITNVLNGPFFWDDYYASEELDRLAESEAKSAVMPVLAKLQGLLTLHVGDAEPKSAEARRRLSFFVNSLFMDVPKAPSISDMMSWTVITPFYSEDVLYNRKDLEAANEDGVNTLLYLQTLYKSDWKNFQERLGLRNDSTSWAGKAKEEIRLWASMRAQTLSRTVQGMMYYEDALHMLSVLDRDPSLMPNAESNSVQQLIKRKFGYVVACQVYGKLKKEQDSKADDIDFLLRRFPSLRVAYIDERQSKSGESSFFSVLIRANDAGTGIEEIYRVRLPGNPVLGEGKPENQNHAMIFSRGEHVQAIDMNQEGYFEDAYKMRNFLQEFALTGSPDMPTTILGFREHIFTGAVSSLANYMALQEYSFVTLGQRVLNRPLRMRLHYGHPDLFDKLFFMQNGGISKASKGINLSEDIFAGYNNLLRGGSVEFKEYVQVGKGRDVGMQQIYKFEAKLSQGAAEQSISRDVYRMVNRVDFFRLLTYYFGGIGHYLSSVLTVAAIWLLVYVLLSLSLFQHEKIGDRPMVPIGTLQIVLAGVGILQTMPLFCALLLERGVWASLTELAQVFISGGPLYFVFHIRTRDYYYSQTILAGGAAYKATGRGFVTQHASFAETFRYFAASHLYLGLEMVAALVLFACYTDAGQYVGRTWSLWFAAVAFLYAPFWFNPMSFEWERVREDVETFVSWMCTTGGSTKNSWESWWKEENGWIKALGPTAKAYLVGRSCIWLVVAAGLLYKPLYLNRKFSGLNYLLFHLGILLGLWQFYRFLDRRGRTRNLPLPYCCTRPTNIVIGMGIVFLVALIIIHSETIKFFVALYYLGAWITVVLSVLGFREQAKIFHWIHDWVLAVVLIIPIFLCTILQFPRHIQTWLLYHNALSQGVVISDLIRHAQNSREMSNTDDERAQAPRSHALASALLNTPSSVNLRSAYSPASGGPMQISPEEKTRERLVGSGGGNGFDTTSGASCKRESFKSGQTRPDHSQSTSQRPHQDPSPVSPAASEQSPEVFQFRQPTNFPTRE

SEQ ID NO: 5 - DNA, 24 bp, Artificial Sequence
  synthetic forward oligonucleotide A92 GlyS LF
aatgcggatgcgttcatggatgtg

SEQ ID NO: 6 - DNA, 24 bp, Artificial Sequence
  synthetic forward nested oligonucleotide A93 GlyS left flank (LF)
tctgcggtccactgacatcatcaa

SEQ ID NO: 7 - DNA, 48 bp, Artificial Sequence
  synthetic reverse oligonucloetide A137 ok299 LF
gaacaacgaacgcaagcgtgtgaatcgatgcccacaatcgaatctcct

SEQ ID NO: 8 - DNA, 48 bp, Artificial Sequence
  synthetic forward oligonucleotide A95 GlyS right flank (RF)
gtgccatcttgttccgtcttgctttggcgttattcgagcgtgagaaga

SEQ ID NO: 9 - DNA, 24 bp, Artificial Sequence
  synthetic reverse nested oligonucloetide A96 GlyS RF
aacattagcaaacgtaaggcggcg

SEQ ID NO: 10 - DNA, 24 bp, Artificial Sequence
  synthetic reverse oligonucleotide A97 GlyS RF
tgcagggcttacctgtatccaaca

SEQ ID NO: 11 - DNA, 4559 bp, Artificial Sequence
  synthetic BGS1 knockout construct
tctgcggtccactgacatcatcaataatgatgagcgggacatgctattgttccatttctttacgggttttgccaaggatatttatttgccagtatttcagacggcgggctcggtggagagggccgcgcggttgtgcgcggagaagggaaaagagttccgcaccttggctgagaaaggaaaggagctccgtgccttggccgatcaggtcgagttgcagatgcagaacgatccaaaccaccaccacaatcagccgtacaggaaggccatcgataacaacaaggcagagatgattaagctggatacggcattgtgggaggagttgtcaaaggataggacgatgcatgaagcagtggcggagacgcttgagttgagcctagaattcttgatgcgcatgcttggggaggatcatgtatcggacgtgaataaggttaagctgacgatggagcgtctgcaggaaagcatgaagggggacgatgcggagaagggaagggcgggggggagaaaggtgatgattttatcggggataaagctggaggaagtggataaggctgtcggagcgttgggcaagatggtcacggcgctgaaaagtgggttgcctcgacgtgtcatcaacccgaaccgcgtcaagcctgcaaagcacacgccgagtgcgcgggagggtcgagggacggtaacggtgggatcggcaatgaagaaggtgcgtagccgcgggtttatgagcaacctctccctctcctcccagaacctcgtggaagtccgggagcaggcggagggccaggcttccgcgtctacgcccacggccagctcgcagcctttacatgagttggacagtttgcgggataaggtgcgggaggcactgagagggtttttgggtgcggtgaaaaagatgttagtgtctggaccgttgtttaaagatgtggcggaagcagtggacaagattttgactggacagtttttttggtgtgatgtgtatgcatccaactccctggatcagttggccaagcctgaggtgaaggaacttgtgcacaagatcctggcgaaactccaaggactcctcaccctgcatgtgggggatgcagagcccaagagtgcggaggcccgtcggcggttgaccttttttgtgaattctttgttcatggatgtgccgaaggcgccctctattgggaatatgttgtcatggacggtagtgacgcctttttattctgaggacgtgctctatagcagaaaggatttggatgcggcgaatgaggacggggtaaaaaccttactgtatctccagacgctgtataaagcggattggaaaaatttccaggagcggttgtcgttgcgggatgatagtccgatttggacggggaaggtgaaagaggagattcgattgtgggcatcgattcacacgcttgcgttcgttgttcttgttttcccctctctaccttcctctcactataaacaaagaaaattttatgtaaaataagggtgacaaaagaagaaccagggagaaaagaaaatgacgggggtaggaaaggactacagagaaaaacatgatgcaggaattcaacactctcatatcaagcaatcagcacaaacaaacgaagacagctacgggagaaaggccttatttctcttccggtaggttaagaagggatggacaatctctcgcgccaacactgagtgctgcggctgctactgctgctgctactgctactaccactggctcttccacagaagcttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatacttaagaagtggtggtggtggtgctgctgctgtagaggatatggcatcgggggtgggacacgagcgggatgtaagtgttgcgatgttttgaggggtttcgtcgggtatggtgcgagtcgtgtgaagatgtggagcacgtgtggaaaagggcaagagaactgggcagaacgtatctaggtttgaaagcactcttcatacttgatcgctggatacgcaactcaagggaaaggtctctcgaaagaacaagagcgagagcccaggctcctagaaggaagagcaaggggaggtctgtccatgtccaatcaggtaaagcacacaaagagcgaagtacaaggtatcagctctagcaacttggtcaactagctgggttttcttgtgacagggaaagactgttgaagatagatcagggggcacttatgggctctcaagagggttgagctgagcctgttccctcgctccgctttgtccgacgacagaaggctttgcgggtcttgccctcggggatccttactgcaaggttgaggcgttgagcagaccccatgggaggtcgttgaggctttcggcactaagacaagataggcaagatgccccaatgtcctgttaccaactggggtgtggaagcacgcctggagcctcaagggctcgttgataaggggatgaaatcgtcccggcgagcaaatcctggttgacctcgcaggatcgttgaaaagcaggaggcacgttcggcgcgagccggtctgttgcagacgcgtgccatcttgttccgtcttgctttggcgttattcgagcgtgagaagataggggatcggccaatggtgcctattggtaccctacaagtggcgcttgctggtgtgggtttgctacagacagccccgctcttttgtgccttactattggagaggggaatttgggctgcgctgacggagctggcgcaggtgtttattagtgggggaccattgtattttgtgtttcatattcgcacacgggattacttttacacgcaaaccattttggcgggtggtgcagcgtatagggcgacggggaggggtttcgtgacgcagcatgcttcttttgcggagacattccggttttttgcgttttcccacctttatttggggctggagatgattgcagccttgattttatttgcgtgtttcacggacgtagggcagtatgtgggtcggacgtggagtttatggtttgcggcgttggcgtttttgtacgcccctttttggtttaatccaatgagttttgagtgggaaagagtgagggaggacttggtgacttttgaggcttggatgcggacaacgggtggctcagcgtcgaactcgtgggaaacttggtggaaggaggagaataagtgggtaaaagagctgaaaaacgtctcggccaggctttatcttgttttgcggtcgtcgatttggttgatggtggcaacggggttgctgtataaacctatcgttgtggatggaaaaatggaccaattgcaatacctgctggagcacctctttgtgttgtttctgctgtttgcgacaagtaactacctggaagggagaagcaggagccgcaaccatcagggtgagtacgcgattatccgtggccttatgattatcctggctataattgcggttagttttttcgtcgtcacggcccagcacacggagacattcaaatttttagtggccctttactacattgccgcctggtgtgccacggtcatgtatgtctccaacagcaagaccgataaccttgtaaaagcctttcacaaagcacacgactggctcctggccacttgctgcttcgtccccataggcatctgcaccataattcagttccccgcctacatccaaacctggctcctctaccacaatgccctctctcaaggcgtcgtcatcggagatcttatccgctacgcgcagaatagtcgggaaaccaccaatatcattgatgaacgcgccgatgcctcctcccttgcgtcaggcttgcctactcctcgttcatccaccatctctttgatgtccggggccaccagagctacaacagctacctctgccgctactaccgtgggaacccttcagatctccccagaggaaaagaccaccgaacgcattgtcgaaattgagggcagcggtgggggcggatataacatactatcccctccgacgggtaccaagaaaaagaatggaaagaatggcacagcctcaaaagcagcgacggaattgccatggcaggcatcggttcaagatgcgcaggatccgtcggtggcagcgccgccgctgcccaatattaacactaacgcggggacggtggagtcgtttcagttccgacagccgaccaattttccgacgcgcgagtgaagggagaagggtgagagggaggaatggagggaggagggagctcgggcaaggcatggttatggatgcagattgatagcgccgccttacgtttgctaatgtt

SEQ ID NO: 12 - DNA, 1388 bp, Artificial Sequence
  synthetic 5′ are of BSG1 KO construct
tctgcggtccactgacatcatcaataatgatgagcgggacatgctattgttccatttctttacgggttttgccaaggatatttatttgccagtatttcagacggcgggctcggtggagagggccgcgcggttgtgcgcggagaagggaaaagagttccgcaccttggctgagaaaggaaaggagctccgtgccttggccgatcaggtcgagttgcagatgcagaacgatccaaaccaccaccacaatcagccgtacaggaaggccatcgataacaacaaggcagagatgattaagctggatacggcattgtgggaggagttgtcaaaggataggacgatgcatgaagcagtggcggagacgcttgagttgagcctagaattcttgatgcgcatgcttggggaggatcatgtatcggacgtgaataaggttaagctgacgatggagcgtctgcaggaaagcatgaagggggacgatgcggagaagggaagggcgggggggagaaaggtgatgattttatcggggataaagctggaggaagtggataaggctgtcggagcgttgggcaagatggtcacggcgctgaaaagtgggttgcctcgacgtgtcatcaacccgaaccgcgtcaagcctgcaaagcacacgccgagtgcgcgggagggtcgagggacggtaacggtgggatcggcaatgaagaaggtgcgtagccgcgggtttatgagcaacctctccctctcctcccagaacctcgtggaagtccgggagcaggcggagggccaggcttccgcgtctacgcccacggccagctcgcagcctttacatgagttggacagtttgcgggataaggtgcgggaggcactgagagggtttttgggtgcggtgaaaaagatgttagtgtctggaccgttgtttaaagatgtggcggaagcagtggacaagattttgactggacagtttttttggtgtgatgtgtatgcatccaactccctggatcagttggccaagcctgaggtgaaggaacttgtgcacaagatcctggcgaaactccaaggactcctcaccctgcatgtgggggatgcagagcccaagagtgcggaggcccgtcggcggttgaccttttttgtgaattctttgttcatggatgtgccgaaggcgccctctattgggaatatgttgtcatggacggtagtgacgcctttttattctgaggacgtgctctatagcagaaaggatttggatgcggcgaatgaggacggggtaaaaaccttactgtatctccagacgctgtataaagcggattggaaaaatttccaggagcggttgtcgttgcgggatgatagtccgatttggacggggaaggtgaaagaggagattcgattgtgggcatcga

SEQ ID NO: 13 - DNA, 1683 bp, Artificial Sequence
  synthetic 3′ are of BSG1 KO construct
tggcgttattcgagcgtgagaagataggggatcggccaatggtgcctattggtaccctacaagtggcgcttgctggtgtgggtttgctacagacagccccgctcttttgtgccttactattggagaggggaatttgggctgcgctgacggagctggcgcaggtgtttattagtgggggaccattgtattttgtgtttcatattcgcacacgggattacttttacacgcaaaccattttggcgggtggtgcagcgtatagggcgacggggaggggtttcgtgacgcagcatgcttcttttgcggagacattccggttttttgcgttttcccacctttatttggggctggagatgattgcagccttgattttatttgcgtgtttcacggacgtagggcagtatgtgggtcggacgtggagtttatggtttgcggcgttggcgtttttgtacgcccctttttggtttaatccaatgagttttgagtgggaaagagtgagggaggacttggtgacttttgaggcttggatgcggacaacgggtggctcagcgtcgaactcgtgggaaacttggtggaaggaggagaataagtgggtaaaagagctgaaaaacgtctcggccaggctttatcttgttttgcggtcgtcgatttggttgatggtggcaacggggttgctgtataaacctatcgttgtggatggaaaaatggaccaattgcaatacctgctggagcacctctttgtgttgtttctgctgtttgcgacaagtaactacctggaagggagaagcaggagccgcaaccatcagggtgagtacgcgattatccgtggccttatgattatcctggctataattgcggttagttttttcgtcgtcacggcccagcacacggagacattcaaatttttagtggccctttactacattgccgcctggtgtgccacggtcatgtatgtctccaacagcaagaccgataaccttgtaaaagcctttcacaaagcacacgactggctcctggccacttgctgcttcgtccccataggcatctgcaccataattcagttccccgcctacatccaaacctggctcctctaccacaatgccctctctcaaggcgtcgtcatcggagatcttatccgctacgcgcagaatagtcgggaaaccaccaatatcattgatgaacgcgccgatgcctcctcccttgcgtcaggcttgcctactcctcgttcatccaccatctctttgatgtccggggccaccagagctacaacagctacctctgccgctactaccgtgggaacccttcagatctccccagaggaaaagaccaccgaacgcattgtcgaaattgagggcagcggtgggggcggatataacatactatcccctccgacgggtaccaagaaaaagaatggaaagaatggcacagcctcaaaagcagcgacggaattgccatggcaggcatcggttcaagatgcgcaggatccgtcggtggcagcgccgccgctgcccaatattaacactaacgcggggacggtggagtcgtttcagttccgacagccgaccaattttccgacgcgcgagtgaagggagaagggtgagagggaggaatggagggaggagggagctcgggcaaggcatggttatggatgcagattgatagcgccgccttacgtttgctaatgtt

SEQ ID NO: 14 - protein, 1443 aa, Artificial Sequence
  synthetic consensus BSG1 polypeptide sequence
CGALLADAAIGCSLCHPALWAQYGDELQLLDHYSPDPAHRQNLGVNILDLRHDAEGMDEELLLMFEQVFGFQTHGVNQVEHLVLLLNQKRYDPALPAGKGPLAPVLHDKFKNYKWCSLKVPHFTIRGGKWFKKMHNLLLLLIWGEAGNRHMPECAWLYHTAACSTQTEVEEEYFLAVTPIYVAVDMKDHKKNYDDFNEFFWSRQCLTWTPDMPAVQAARKAGEGEGLILKPKTFEKRSWLMIMLAFRRLIDFHVVTFFLAQGFWLNLQWDDPYYQMSVFLMNLGIWLEWQCFREAKHGVMLRLLRFVFLFQGLSLGLDLPLSKSVQLEWWMYVWISVALHVWCVFQPLSTVFERNHYVKALLDIFPQRYTGKRVEPFKWYFWVKIFSYQFEVPLALPALELADDQNLNNYLTILIRWLPFVAIYLDMIIYSLAGVGVGLELGQVDFAGIREFMRTPESFSRLINDRKSRKASVSDLGMSRRFTSRNLAAADERPLAGMQGGGHNDAFDGTTKWAFTAWNKVNLRTDIINDERDMLLFFFGKIYLPVFQTAGVEAARLCAEKGKEFRLNDHQDKDLELKDRTEAVAETLELLFLRMLGHDVTEQSEGILLEVKAVALGKVALKSGLPRRVINPRVPKREGVTVGSMVRSGFMSNLSLSSQLVVEQAAPQPELDSLRDKRLRFLVKVGLDVAALGFFWDYASLDLAEKVLAKLQGLLTLHVGDAEPKSAEARRRLFFVNSLFMDVPKAPSIMSWTVTPFYSEDVLYRKDLAANEDGVTLLYLQTLYKDWKNFQERLLRDSWGKKEEIRLWASMRAQTLSRTVQGMMYYEDALHLSLDDENQLIRKFGYVVACQVYGKLKKEQDSKADDIDFLLRFPLRVAYIDEQSKSGESFSVLIRADIEEIYRVRLPGNPLGEGKPENQNHAMIFSRGEHVQAIDMNQEGYFEDAKMRNFLQEFATGPDMPTTILGFREHIFTGASSLANYMALQEYSFVTLGQRVLNRPLRMRLHYGHPDLFDKLFFQNGGISKASKGINLSEDIFAGYNNLLRGGSVEFKEYVQVGKGRDVGMQQIYKFEAKLSQGAAEQSISRDVRMRVDFFRLLYYFGGIGHYLSSVLTVAAIWLLVYLLLLFEKIGDRPMVPIGTLQLAGVGLQTPLFCALLLERGWALTELAQVFISGGPLYFVFHIRTRDYYQTILAGGAAYATGRGFVTQHASFAETFRFASHLYLGLEMAALLFACTDGQYVGRTWSLWFAAAFLYAPFWFNPMSFEWERVREDTFWMTTGGSNSWEWWKEENWKLAYLVRSIWLVAGLLYKPKLYLLHLLLLRRRNYRIVHETKFVALYYAWVVKFHHDWLAPICTIQFPIQTWLLYHNALSQGVVIDLIRAQNSRENDERAALASLTPSLSAGQISPEEKTERVGSGGGKKGSQDPSPEFQFRQPTNFPTRE

(19) United States
(12) Patent Application Publication
(10) Pub. No.: US 2016/0017352 A1
(43) Pub. Date: Jan. 21, 2016

(54) ALGAL STRAIN WITH REDUCED BETA GLUCAN SYNTHASE ACTIVITY

(71) Applicant: Aurora Algae, Inc., Hayward, CA (US)

(72) Inventor: OLIVER KILIAN, Castro Valley, CA (US)

(21) Appl. No.: 14/737,218

(22) Filed: Jun. 11, 2015

Related U.S. Application Data
(60) Provisional application No. 62/025,457, filed on Jul. 16, 2014.

(51) Int. Cl. C12N 15/82; C12P 7/64

(52) U.S. Cl. CPC C12N 15/8247; C12P 7/64

(57) ABSTRACT

The present invention provides compositions of a modified algal cell and methods of making thereof. In particular, the modified cell has suppressed expression or activity of endogenous beta glucan synthase 1 (BGS1) and increased lipid synthesis when grown under nutrient deficient conditions.

### RELATED APPLICATIONS

This application claims priority to U.S. Provisional Patent Application No. 62/025,457, filed on Jul. 16, 2014, the contents of which are incorporated by reference in the entirety for all purposes.

### BACKGROUND OF THE INVENTION

Polysaccharides that are believed to have originated from cell walls or from storage polysaccharides in the eustigmatophyte Monodus subterranus have been identified as a beta-D-glucan containing both 1,3- and 1,4-linked units. Beta glucan polysaccharides represent major carbohydrate polysaccharides in protozoans and chromista. Beta 1,3 glucans are associated with storage polysaccharides and also have structural functions. Beta 1,4 glucans are mainly components of structural polysaccharides, such as cell walls. Beta 1,3 glucan storage carbohydrates have been described in euglenoids as paramylon; in diatoms, haptophytes and chrysophytes as chrysolaminarin; in brown algae as laminarin; and in oomycetes as mycolaminarin. In addition, other structural beta glucan are found in components of cell walls in many protozoans and chromists, such as callose (a 1,3-β-glucan), cellulose (a 1,4-β-glucan), chitin, (a 1,4-β-N-acetylglucosamine glucan), and (1,3:1,4)-β-glucans.

1,3-Beta-glucan synthase (EC. 2.4.1.34), also known as callose synthase, is a glucosyltransferase enzyme involved in the generation of beta-glucan in organisms such as fungi.

In photosynthetic organisms such as plants and algae, once inorganic carbon is fixed, various biosynthesis pathways such as protein biosynthesis, storage and structural polysaccharide biosynthesis, and lipid biosynthesis compete as sinks for the organic carbon. For instance, a decreased flux into the polysaccharide biosynthesis pathway may increase the activity of the lipid biosynthesis pathway.

There remains a need to increase the accumulation of lipids in algal cells, which can be used in the production of nutriceuticals, feedstock, and biofuels. The present invention addresses these needs and provides additional advantages by providing modified algal cells with increased lipid synthesis and diminished carbohydrate synthesis.

### BRIEF SUMMARY OF THE INVENTION

In one aspect, the present invention provides a modified algal cell having (1) suppressed expression or activity of endogenous beta glucan synthase 1 (BGS1), such as an endogenous BGS1 gene, RNA transcript or protein; and (2) increased lipid synthesis when grown under nutrient deficient conditions, such as nitrogen starvation. When grown under nutrient deficient conditions, the modified algal cell can have decreased sugar content compared to a wild-type algal cell (e.g., a wild-type cell of the same genus). In addition, such a modified algal cell can have at least 50% less sugar content compared to a wild-type cell. When grown under nutrient deficient conditions, the algal cell can have at least 25% more lipid content compared to a wild-type cell. The modified algal cell can have at least 40% lipid content by ash-free dry weight.

In some embodiments, suppressed expression or activity of endogenous BGS1 includes contacting the algal cell with an inhibitor of BGS1. The inhibitor of BGS1 can be a siRNA, a microRNA, or an antisense RNA. In other embodiments, suppressed expression or activity of endogenous BGS1 includes inactivation or removal of the endogenous BGS1 gene by gene editing.

In yet other embodiments, suppressed expression or activity of endogenous BGS1 includes inactivation or removal of the endogenous BGS1 gene by homologous recombination. The endogenous BGS1 gene can include the nucleic acid sequence set forth in SEQ ID NO: 1 or SEQ ID NO: 3. In some instances, the step of inactivating, interrupting, or removing the BGS1 gene comprises inserting a selectable marker into the BGS1 gene. The selectable marker that is inserted into the BGS1 gene can replace a portion of the BGS1 gene.

The modified algal cell is of the genus Nannochloropsis. In some instance, Nannochloropsis is selected from the group consisting of Nannochloropsis gaditana, Nannochloropsis granulate, Nannochloropsis limnetica, Nannochloropsis oceanica, Nannochloropsis oculata and Nannochloropsis salina. The algal cell can be an auxotroph.

In another aspect, the present invention provides a method for making the algal cell described above. The method includes (a) transforming the algal cell with a targeting construct comprising a selectable marker, wherein the selectable marker is flanked at the 5′ end by a first nucleic acid sequence of an endogenous BGS1 gene and at the 3′ end by a second nucleic acid sequence of the endogenous BGS1 gene, and wherein said targeting construct integrates into the algal nuclear genome by homologous recombination, thereby inactivating, interrupting or removing the endogenous BGS1 gene; and (b) selecting the transformed algal cell carrying the inactivated BGS1 gene, thereby suppressing the expression of BGS1. The endogenous BGS1 gene can include the BGS1 promoter or one or more regulatory elements. The first nucleic acid sequence of the endogenous BGS1 gene can be about 200 bp to about 5 kb of the BGS1 gene. The second nucleic acid sequence of the endogenous BGS1 gene can be about 200 bp to about 5 kb of the BGS1 gene. The first and second nucleic acid sequences can be different lengths. In other embodiments, the first and second nucleic acid sequences are the same lengths. The first and second nucleic acids sequences can be non-overlapping sequences of the BGS1 gene. The selectable marker of the targeting construct can be an antibiotic resistance gene. In some instances, the antibiotic resistance gene is a zeocin-resistance gene, a blasticidin-resistance gene, or a hygromycin-resistance gene. The selectable marker can also include a promoter, such as a heterologous promoter. The promoter can be the acyl carrier protein (ACP) promoter or a fragment thereof. In some instances, the promoter is a bidirectional promoter. The promoter can be the violaxanthin-chlorophyll a binding protein (VCP) bidirectional promoter or a fragment thereof. The selectable marker can replace a portion of the endogenous BGS1 gene.

In some embodiments, the step of suppressing the expression or the activity of endogenous BGS1 includes contacting the algal cell with an inhibitor of BGS1, such as a siRNA, microRNA or an antisense RNA.

The algal cell can be of the genus Nannochloropsis. The Nannochloropsis can be selected from the group consisting of Nannochloropsis gaditana, Nannochloropsis granulate, Nannochloropsis limnetica, Nannochloropsis oceanica, Nannochloropsis oculata and Nannochloropsis salina. The algal cell can be a wild-type cell or an auxotroph.

In a third aspect, the present invention provides a method for obtaining at least 40% lipids by ash-free dry weight from an algal biomass derived from an algal cell grown under a nutrient deficient condition, such as nitrogen starvation. The method includes (a) cultivating any one of the algal cells described above, under the nutrient deficient condition; (b) generating an algal biomass from the cells; and (c) extracting lipids from the algal biomass, wherein the lipid content (amount) is at least about 40% lipids per ash-free dry weight.

Other objects, features, and advantages of the present invention will be apparent to one of skill in the art from the following detailed description and figures.

### BRIEF DESCRIPTION OF THE DRAWINGS

FIG. 1 shows the nucleotide sequence of the Nannochloropsis oceanica W2J3B BGS1 gene.

FIG. 2 shows the amino acid sequence of the Nannochloropsis oceanica W2J3B BGS1 polypeptide.

FIG. 3 shows the Pfam motif localization with the BGS1 polypeptide. Amino acids 1299-1644 are eliminated in the BGS1 knockout algal strain.

FIG. 4 shows a list of homologues of the Nannochloropsis W2J3B BGS1 polypeptide identified in other algal groups.

FIG. 5 shows the nucleotide sequence of the Nannochloropsis gaditana (IC164 isolate) BGS1 gene.

FIG. 6 shows the amino acid sequence of the Nannochloropsis gaditana (IC164 isolate) BGS1 polypeptide.

FIG. 7 shows an amino acid sequence comparison between the Nannochloropsis oceanica W2J3B BGS1 polypeptide and the Nannochloropsis gaditana (IC164 isolate) BGS1 polypeptide.

FIG. 8 shows the oligonucleotides used to generate the BGS1 targeting construct.

FIG. 9 shows the nucleic acid sequence of the BGS1 targeting construct.

FIG. 10 shows the percent lipid content per ash-free dry weight of the BGS1 knockout mutant algal cells and wild-type cells of the parental strain.

FIG. 11 shows the lipid content per culture volume of the BGS1 knockout mutant algal cells and wild-type cells during culturing in nutrient deficient medium.

FIG. 12 shows the sugar and lipid contents per ash-free dry weight of the BGS1 knockout mutant algal cells, normalized to contents in the wild-type cells.

FIG. 13 shows the cell mass composition of wild-type cells and BGS1 knockout mutant algal cells at a nutrient sufficient condition (d0) and a nutrient deficient condition (d1).

### DETAILED DESCRIPTION OF THE INVENTION

### I. INTRODUCTION

The present invention provides methods for increasing lipid synthesis in an algal cell. The invention is based, in part, on the discovery that disruption of beta glucan synthase expression and/or activity in an algal cell results in the accumulation of lipids and the reduction of carbohydrates in the absence of nutrients such as nitrogen. For instance, the modified (e.g., non-naturally occurring) algal cell can accumulate at least about 50% less glucose units per cell compared to a wild-type cell. When grown in the nutrient deficient conditions, the algal cell has at least about 25% more lipid content per ash-free dry weight compared to that of a wild-type cell.

### II. DEFINITIONS

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

In this disclosure the term “or” is generally employed in its sense including “and/or” unless the content clearly dictates otherwise.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “expression” when referring to a gene, is used to mean the transcription of a DNA to form an RNA molecule encoding a particular protein (e.g., algal BGS1 protein) or the translation of a protein encoded by a polynucleotide sequence. In other words, both mRNA level and protein level encoded by a gene of interest (e.g., algal BGS1 gene) are encompassed by the term “gene expression level” in this disclosure.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “beta glucan synthase 1 gene,” “BGS1 gene,” “beta glucan synthase 1 protein,” or “BGS1 protein” refers to any naturally occurring variants or mutants, interspecies homologs, or man-made variants of the algal BGS1 gene or BGS1 protein. “Endogenous beta glucan synthase 1 gene” or “endogenous BGS1 gene” refers to the manually occurring BGS1 gene of a specific cell or organism.

“Inhibitor” of BGS1 is used to refer to inhibitory molecules or agents that, e.g., partially or totally block, decrease, prevent, delay activation, inactivate, or down regulate the activity of BGS1 mRNA or protein. An “inhibitor” can have the ability of negatively affecting the level or activity of BGS1 mRNA or protein by at 10%, preferably, at least 20%, 30%, 40%, 50%, 60%, 70% or higher, compared to the level of BGS1 mRNA or protein in the absence of the inhibitor.

The term “transforming” refers to introducing DNA such as exogenous DNA inside the cell wall of a cell. The exogenous DNA can integrate (e.g., become covalently linked) to the chromosomal genomic DNA of the cell. Alternatively, the exogenous DNA can be maintained on an extrachromosomal element, such as a plasmid. A daughter cell of a transformed cell can inherit the exogenous DNA through chromosome replication.

The term “targeting construct” refers to a vector contains an insertion cassette flanked by regions of homology to an insertion site, the insertion cassette containing a polynucleotide to be inserted at the insertion site during homologous recombination. Transformation of a cell with the targeting construct can provide a cell in which an endogenous nucleic acid or portion thereof is replaced by the insertion cassette or a portion thereof. In some cases, the insertion cassette contains a modified version of the endogenous nucleic acid or a portion thereof. In some cases, the insertion cassette contains a selectable marker or a heterologous nucleic acid. In some cases, the insertion cassette contains a polynucleotide operably linked to a promoter and is thus also an expression cassette. In some cases, insertion of the insertion cassette, or a portion thereof, at a site adjacent to, or near, an endogenous promoter can provide for expression of a polynucleotide in the insertion cassette.

The term “promoter” refers to a DNA regulatory region capable of binding RNA polymerase in a cell and initiating transcription of an associated heterologous polynucleotide, e.g., coding sequence. A coding sequence is “under the control” of the promoter sequence when RNA polymerase which binds the promoter sequence will transcribe the coding sequence into mRNA, which is then in turn translated into the protein encoded by the coding sequence. The promoter sequence is bounded at its 3′ terminus by the translation start codon of a coding sequence and extends upstream (5′ direction) to include at least the minimum number of bases or elements necessary to initiate transcription at levels detectable above background. Promoters may contain additional consensus sequences (promoter elements) for more efficient initiation and transcription of downstream genes.

The term “operably linked” refers to a configuration in which a regulatory sequence is placed at an appropriate position relative to a polynucleotide sequence such that the regulatory sequence affects or directs expression of the polynucleotide sequence, for example, to produce a polypeptide and/or functional RNA. Thus, a promoter is operably linked to a nucleic acid sequence (e.g., a gene) such that it can mediate transcription of the nucleic acid sequence.

The term “selectable marker cassette” refers to a polynucleotide sequence (e.g., gene) that confers a phenotype on a cell in which it is expressed to facilitate the selection of cells that are transfected or transformed with a targeting construct of the present invention. In some instances, the selectable marker cassette includes a promoter that drives the expression of the selectable marker gene. Non-limiting examples of a selectable marker include genes conferring resistance to antibiotics, such as amikacin (aphA6), ampicillin (amp), blasticidin (bis, bsr, bsd), bleomicin or phleomycin (ZEOCIN™) (ble), chloramphenicol (cat), emetine (RBS 14p or cry 1-1), erythromycin (ermE), G418 (GENETICIN™) (neo), gentamycin (aac3 or aacC4), hygromycin B (aphlV, hph, hpt), kanamycin (nptll), methotrexate (DHFR mtxR), penicillin and other β-lactams (β-lactamases), streptomycin or spectinomycin (aadA, spec/strep), and tetracycline (tetA, tetM, tetQ); genes conferring tolerance to herbicides, such as genes conferring tolerance to herbicides such as aminotriazole, amitrole, andrimid, aryloxyphenoxy propionates, atrazines, bipyridyliums, bromoxynil, cyclohexandione oximes dalapon, dicamba, diclfop, dichlorophenyl dimethyl urea (DCMU), difunone, diketonitriles, diuron, fluridone, glufosinate, glyphosate, halogenated hydrobenzonitriles, haloxyfop, 4-hydroxypyridines, imidazolinones, isoxasflutole, isoxazoles, isoxazolidinones, miroamide B, p-nitrodiphenylethers, norflurazon, oxadiazoles, m-phenoxybenzamides, N-phenyl imides, pinoxadin, protoporphyrionogen oxidase inhibitors, pyridazinones, pyrazolinates, sulfonylureas, 1,2,4-triazol pyrimidine, triketones, urea; acetyl Co A carboxylase (ACCase), acetohydroxy acid synthase (alias), acetolactate synthase (als, csrl-1, csrl-2, imrl, imr2), aminoglycoside phosphotransferase (apt), anthranilate synthase, bromoxynil nitrilase (bxn), cytochrome P450-NADH-cytochrome P450 oxidoreductase, dalapon dehalogenase (dehal), dihydropteroate synthase (sul), class I 5-enolpyruvylshikimate-3-phosphate synthase (EPSPS), class II EPSPS (aroA), non-class III EPSPS, glutathione reductase, glyphosate acetyltransferase (gat), glyphosate oxidoreductase (gox), hydroxyphenylpyruvate dehydrogenase, hydroxy-phenylpyruvate dioxygenase (hppd), isoprenyl pyrophosphate isomerase, lycopene cyclase, phosphinothricin acteyl transferase (pat, bar), phytoene desaturase (crtJ), prenyl transferase, protoporphyrin oxidase, the psbA photosystem II polypeptide (psbA), and SMM esterase (SulE) superoxide dismutase (sod); and genes that may be used in auxotrophic strains or to confer other metabolic phenotypes, such as arg7, his3, hisD, hisG, lysA, manA, metE, nitl, trpB, ura3, xylA, a dihydrofolate reductase gene, a mannose-6-phosphate isomerase gene, a nitrate reductase gene, or an ornithine decarboxylase gene; a negative selection factor such as thymidine kinase; or toxin resistance factors such as a 2-deoxyglucose resistance gene.

The term “homologous recombination” refers to an exchange of homologous polynucleotide segments anywhere along a length of two nucleic acid molecules. Homologous recombination includes the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. For a description of the enzymes and mechanisms involved in homologous recombination, see, for example, Watson et al, “Molecular Biology of the Gene,” pages 313-327, The Benjamin/Cummings Publishing Co. 4th ed. (1987).

The term “RNAi” refers to RNA interference strategies of reducing expression of a targeted gene. RNAi techniques employ genetic constructs within which sense and anti-sense sequences are placed in regions flanking an intron sequence in proper splicing orientation with donor and acceptor splicing sites. Alternatively, spacer sequences of various lengths can be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes. The phenomenon of RNA interference is described and discussed in Bass, Nature 411: 428-29 (2001); Elbahir et al., Nature 411: 494-98 (2001); and Fire et al., Nature 391: 806-11 (1998); and WO 01/75164, where methods of making interfering RNA also are discussed.

A “short hairpin RNA” or “small hairpin RNA” is a ribonucleotide sequence forming a hairpin turn which can be used to silence gene expression. After processing by cellular factors the short hairpin RNA interacts with a complementary RNA thereby interfering with the expression of the complementary RNA.

Two nucleic acid sequences or polypeptides are said to be “identical” if the sequence of nucleotides or amino acid residues, respectively, in the two sequences is the same when aligned for maximum correspondence as described below. The terms “identical” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988).

The term “substantial identity” of polynucleotide sequences means that a polynucleotide comprises a sequence that has at least 25% sequence identity. Alternatively, percent identity can be any integer from at least 25% to 100% (e.g., at least 25%, 26%, 27%, 28%, 29%, 30%, 31%, 32%, 33%, 34%, 35%, 36%,37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%), preferably calculated with BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from at least 40% to 100% (e.g., at least 40%, 41%, 42%, 43%, 44%, 45%, 46%, 47%, 48%, 49%, 50%, 51%, 52%, 53%, 54%, 55%, 56%, 57% 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). More preferred embodiments include at least 60%, 65%, 70%, 75%, 80%, 85%, 90%, 95%, or 99%. The present invention provides polynucleotides substantially identical to the beta glucan synthase 1 gene of Nannochloropsis spp. (SEQ ID NO:1). The present invention also provides polypeptides and polynucleotides encoding such polypeptides) substantially identical to the beta glucan synthase 1 polypeptide of Nannochloropsis spp. (SEQ ID NO:2). Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are valine-leucine-isoleucine, phenylalanine-tyrosine, lysine-arginine, alanine-valine, aspartic acid-glutamic acid, and asparagine-glutamine.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to as segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Unless otherwise indicated, the comparison window extends the entire length of a reference sequence. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

As used in this application, an “increase” or a “decrease” refers to a detectable positive or negative change in quantity from a comparison control, e.g., an established standard control (such as an average lipid content or sugar content in a modified algal cell). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term “ash-free dry weight” or “AFDW” refers to a measurement of the weight of an organic material that is substantially free of water. It may be the dry weight of the organic content (and not the inorganic content) of a sample. In some instances, matter to be weighed is collected on an ashed filter, dried and weighed. The dried material can be oxidized (e.g., ashed) at a high temperature and reweighed. The loss of weight upon oxidation is referred to as the ash-free dry weight.

As used herein, the following terms have the meanings ascribed to them unless specified otherwise.

### III. DETAILED DESCRIPTIONS OF EMBODIMENTS

The invention is based, in part, on the discovery of a beta glucan synthase (BGS1) gene and corresponding polypeptide in the eustigmatophyte Nannochloropsis. Using homologous recombination technology, the inventors have disrupted the BGS1 gene. The modified algal cell when cultured in the absence of nutrients, such as nitrogen can accumulate lipids faster with respect to a wild-type cell (e.g., a parental cell). In addition, the modified algal cell can have less sugar content compared to a wild-type cell. Thus, algal biomass derived from such an algal cell is enriched in lipids and reduced in carbohydrate content compared to that of a wild-type cell.

A. General Methodology

Practicing this invention utilizes routine techniques in the field of molecular biology. Basic texts disclosing the general methods of use in this invention include Sambrook and Russell, Molecular Cloning, A Laboratory Manuel (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

B. Algal Beta Glucan Synthase 1

The algal BGS1 gene can have at least 85% identity, e.g., at least 85%, 86%, 87%, 88%, 89%, 90%, 91%, 92%, 93%, 94%, 95%, 96%, 97%, 98%, 99% or 100% identity to the nucleic acid sequence of SEQ ID NO: 1 or SEQ ID NO:3 The BGS1 gene can encode an algal BGS1 polypeptide having at least 85% identity, e.g., at least 85%, 86%, 87%, 88%, 89%, 90%, 91%, 92%, 93%, 94%, 95%, 96%, 97%, 98%, 99% or 100% identity to the amino acid sequence of SEQ ID NO: 2 or SEQ ID NO: 4. The BGS1 gene can encode an algal BGS1 polypeptide having at least 85% identity, e.g., at least 85%, 86%, 87%, 88%, 89%, 90%, 91%, 92%, 93%, 94%, 95%, 96%, 97%, 98%, 99% or 100% identity to the amino acid sequences of NCBI Reference Sequence Nos. XP_{—}002177443.1, XP_{—}002177442.1, EBZ28172.1, CCA25481.1, XP_{—}002906408.1, EJK49176.1, XP_{—}002294317.1, EGB08046.1, DAA43105.1, EGZ28309.1, XP_{—}003532149, NP_{—}001048628.1, and ACS36248.1.

C. Methods for Suppressing Expression or Activity of BGS1

The invention relates to inactivating or interrupting the endogenous BGS1 gene or suppressing the activity of BGS1 RNA in an algal cell. The modified algal cell can be cultured under nutrient deficient conditions to increase its lipid content and decrease its sugar content. Thus the first steps of practicing the invention are to generate an algal cell with suppressed expression or activity of BGS1.

The BGS1 gene of the cell (including the codimg sequence as well as its upstream and/or downstream non-coding regulatory sequences, e.g., the promoter region) can be modified by homologous recombination. The targeting construct for homologous recombination can be made according to standard molecular biology methods known to those skilled in the art. The construct can contain a nucleic acid sequence that includes a portion of the BGS1 gene encoding the BGS1 polypeptide. In some embodiments, the construct contains a nucleic acid sequence that is adjacent to the BGS1 gene in the host cell genome. The BGS1 gene of the construct can include at least one variant/mutation that corresponds to at least one amino acid substitution, deletion, insertion or addition to the wild-type BGS1 polypeptide.

The targeting construct can include two nucleic acid sequences (e.g., the 5′ and 3′ homologous arms) that are homologous to the BGS1 gene including the adjacent region of the genome to be modified and a selectable marker. The homologous region in the host genome is disrupted by the insertion of a foreign sequence, such as the selectable marker that allows selection with the construct integrated into the host cell genome. The selectable marker in the construct can be flanked by the 5′ and 3′ homologous arms.

In some embodiments, the 5′ homologous arm or the 3′ homologous arm of the targeting construct is about 1000 bps in length. The 5′ homologous arm or the 3′ homologous arm of the targeting construct can be about less than 1000 bps, e.g., 950 bps, 900 bps, 850 bps, 800 bps, 750 bps, 700 bps, 650 bps, 600 bps, 550 bps, 500 bps, 450 bps, 400 bps, 350 bps, 300 bps, 250 bps, 200 bps, 150 bps, 100 bps, or less, in length. The 5′ homologous arm or the 3′ homologous arm of the targeting construct can be greater than 1000 bps, e.g., 1100 bps, 1200 bps, 1300 bps, 1400 bps, 1500 bps 1600 bps, 1700 bps, 1800 bps, 1900 bps, 2000 bps, 2500 bps, 3000 bps, 3500 bps, 4000 bps, 5000 bps, 6000 bps, 7000 bps, 8000 bps, 9000 bps, 10000 bps or more, in length. The 5′ and 3′ homologous arms can be the same length. Alternatively, the 5′ and 3′ arms are different lengths.

The selectable marker can be an antibiotic resistance gene. Such a gene can confer antibiotic resistance to any host cell that carries the genome-integrated targeting construct. Non-limiting examples of an antibiotic resistance gene include genes that confer resistance to ampicillin, phleomycin, paramomycin, neomycin, spectinomycin, streptomycin, G418, amikacin, kanamycin, chloramphenicol, zeocin, bleomycin, hygromycin B, blasticidin, and the like, and combinations thereof. Gene expression of the selectable marker can be control by operably linking a promoter to the antibiotic resistance gene. For instance, a violaxanthin-chlorophyll a binding protein (Vcp2) promoter (see, U.S. Pat. No. 8,318,482, the disclosure is hereby incorporated by reference in its entirety for all purposes) can be used to drive high levels of gene expression in algal cells at low light intensities. The Vcp2 promoter can be operably linked to, for example, the Sh ble gene found in Streptoalloteichus hindustanu, the hygromycin B phosphotransferase gene, or the blastocidin S deaminase gene. In some embodiments, the selectable marker also contains a 3′UTR, such as a Vcp1 3′UTR positioned downstream of the market gene. In other embodiments, the acyl carrier protein (ACP) promoter can be used to drive gene expression in algal cells. Detailed description of the ACP promoter is found in, e.g., U.S. Patent Application Publication No. 2013/0289262, the contents are herein incorporated by reference in its entirety for all purposes. Non-limiting examples of useful promoters include the cauliflower mosaic virus promoter 35S (CaMV35S), the SV40 promoter, the ribulose bisphosphate carboxylase, small subunit (RBCS2) promoter, the abundant protein of photosystem I complex (PsaD) promoter, the HSP70A/RBCS2 promoter, the HSP70A/β2 tubulin promoter, and the like.

Additional the selectable markers include fluorescent or chromogenic markers such as, but not limited to, luciferase, β-glucoronidase, β-galactosidase, green fluorescent protein, and variant thereof. Herbicide-based selectable markers, such as the gene for acetolactate synthase that confers resistance to sulphonylurea herbicides or the pds gene that confers resistance to fluorochloridane can be used.

In some embodiments, the targeting construct comprises the nucleic acid sequence of SEQ ID NO:11.

The targeting construct can be introduced into the algal genome by any method known in the art, such as agitation in the presence of glass beads or silicon carbide whiskers, electroporation, or bombardment of DNA binding particles using a particle gun. See, U.S. Pat. No. 8,759,615, the disclosure is hereby incorporated in its entirety for all purposes.

Algal cells that have undergone homologous recombination with the target construct of the present invention to suppress the expression of the BGS1 gene can be verified by using standard molecular biology techniques, such as PCR and Southern blot analysis.

In some embodiments, the BGS1 gene is inactivated by gene editing, e.g., causing double-stranded breaks within or surrounding the gene by contacting the genomic DNA with one or more agents capable of cleaving the DNA. For instance, the gene editing agent can recognize and/or bind to a specific polynucleotide recognition sequence within or near the BGS1 gene to produce a break at or near the recognition sequence. Examples of such an agent include, but are not limited to, endonucleases, site-specific recombinases, transposases, topoisomerases, meganucleases, Cas9 nucleases of the CRISPR/Cas systems (see, U.S. Pat. No. 8,697,359) a TAL-effector DNA binding domain-nuclease fusion proteins (TALENs; see, e.g., Gaj et al., Trends Biotechnol, 31:397-405, 2013), and zinc finger nucleases, and include modified derivatives, variants, and fragments thereof.

An algal cell with suppressed expression of BGS1 (e.g., DNA) can be created in vitro using other genetic engineering techniques, such as site directed mutagenesis, oligonucleotide directed mutagenesis, random chemical mutagenesis, Exonuclease III deletion procedures, and standard cloning techniques.

Methods for suppressing BGS1 (e.g., RNA) activity include reducing the amount or stability of mRNA by using RNAi, microRNA, shRNA, siRNA, antisense RNA, and ribozyme constructs. The algal cell can be transformed with an RNAi, microRNA, shRNA, siRNA, antisense RNA, or ribozyme construct that targets BGS1 mRNA using methods known in the art. Detailed descriptions of methods for using antisense RNA or RNAi in algal cells are found in, e.g., Shroda et al., The Plant Cell, 11:1165-78, 1999; Ngiam et al., Appl. Environ. Microbiol., 66:775-782, 2000; Ohnuma et al., Protoplasma, 236:107-112, 2009; Lavaud et al., PLoS One, 7:e36806, 2012; Cerruti et al., Eukaryotic Cell, 10: 1164-1172 (2011); and Shroda et al., Curr Genet., 49: 69-84, 2006). Detailed descriptions of ribozyme constructs are found in, e.g., Haseloff et al., Nature, 334:585-891, 1988.

For example, a nucleic acid sequence of the BGS1 polynucleotide can be operably linked to a promoter such that the antisense strand of the RNA is transcribed. The nucleic acid sequence can be from about 25 bps to about 3 kilobases or more in length, e.g., from about 25 bps to about 50 bps, from about 500 bps to about 1 kb, from about 1 kb to about 2 kb, or from about 2 kb to about 4 kb in length.

In some embodiments, a double stranded RNA that is substantially identical to the BGS1 polynucleotide (or a fragment thereof) or complementary thereof is introduced or produced by the algal cell by expression, for example, of an RNAi construct, such as a short hairpin RNA (shRNA) construct. The RNAi construct can include a nucleic acid sequence that has at least 70% identity, e.g., at least 70%, 75%, 80%, 85%, 90% 95%, 99% or 100% identity to the BGS1 polynucleotide.

Suppressing BGS1 activity can results in decreased levels or undetectable levels of the BGS1 polypeptide. In some embodiments, the algal cell of the present invention has low or undetectable levels of a polypeptide with at least 60%, e.g., at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 90%, or 100% identity to the amino acid sequence of SEQ ID NOs: 2 or 4.

D. Culturing Cells Under Nutrient Deficient Conditions

Algae can be cultured under conditions to promote the accumulation of lipids and the reduction of sugar in the cells. For instance, the lipid content and compositions can be modulated by varying growth conditions such as light intensity, light-dark cycles, temperature, nutrient content, nutrient availability, salinity, pH, culture density, culture temperature, and other environmental conditions. Descriptions of growth conditions for Nannochloropsis are found in, e.g., Sukenik, A. “Chapter 3: Production of eicosapentaenoic acid by the marine Eustigmatophyte Nannochloropsis,” Chemicals from Microalgae., ed. Zvi Cohen, CRC Press, 1999, and Pal et al., Appl Microbiol Biotechnol, 2011, 90:1429-1441. Standard culture systems such as open ponds, e.g., open race way ponds, and photobioreactors can be used to grow algae. The modified algal cells can be cultured in solid or liquid growth media. Recipes and formulations for making growth media are known by those skilled in the art, as are instructions for the preparation of particular media suitable for algal cells. For example, useful fresh water and salt water media can include those described in Barsanti (2005) Algae: Anatomy, Biochemistry & Biotechnology, CRC Press for media and methods for culturing (cultivating) algae. Algal media recipes can also be found from, for example, the UTEX Culture Collection of Algae at the University of Texas, the Culture Collection of Algae and Protozoa, and the CAUP Culture Collection of Algae.

In some embodiments, the nutrient content in the media is deficient or deplete, such that, the amount of one or more essential growth nutrients is supplied at a growth limiting amount. The growth limiting nutrient can include compounds containing nitrogen, phosphorus, sulfig, molybdenum, magnesium, cobalt, nickel, silicon, iron, zinc, copper, potassium, calcium, boron, chlorine, sodium, selenium, specific vitamins and any other compounds that may be essential for propagation of an algal cell or culture. In some embodiments, the modified algal cell is cultured under nutrient deficient conditions, such as under nitrogen deficient, deprivation, limiting, or depleted conditions. For instance, the algal cell can be grown in culture media lacking nitrogen.

To generate an algal biomass, standard methods, e.g., flocculation, centrifugation, and filtration (dead end filtration, microfiltration, ultrafiltration, pressure filtration, and tangential flow filtration) can be used for dewatering algae. For instance, cationic chemical flocculants, such as Al₂(SO₄)₃, FeCl₃, and Fe₂(SO₄)₃, can be used to coagulate harvested algae into a biomass.

E. Methods for Measuring Lipid and Sugar Content in Algal Cells

The lipid content of the algal cell or algal biomass can be determined using standard methods recognized by those in the art. In some embodiments, the lipid content is measured by direct trans-esterification and subsequent gas chromatography analysis. For example, the lipids can be measured by transesterifying all free and ester-linked fatty acids to fatty acid methyl esters (FAMEs) in as solution of methanol and toluene, using hydrochloric acid as a catalyst. The FAMEs can be extracted from the reaction mixture with hexanes, then concentrated and analyzed on, for example, an Agilent 6890 gas chromatograph equipped with a 30 m×0.25 mm×0.25 μm capillary column coated with a polyethylene glycol stationary phase (USP G16). Quantification can be done relative to ethyl tricosanoate used as an internal standard. Fatty acid ethyl esters can be measured using AOCS Official Method Ce 1b-89 (Fatty Acid Composition of Marine Oils by GLC). In vivo measurements of lipid content can be made by using lipophilic dyes such as Nile Red or BODIPY.

In some embodiments, the lipid content of the modified algal cell of the present invention has the similar or the same lipid content (% per ash-free dry weight) as a wild-type or control cell when cultured under nutrient rich conditions. In some embodiments, the lipid content (% per ash-free dry weight) of the modified cell is at least about 20%, e.g., at least about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more, higher, than that of a wild-type cell when cultured under nutrient deficient conditions (e.g., without nitrogen). In some instances, the modified algal cell can accumulate more lipid per culture volume compared to a wild-type cell.

The modified algal cell when cultured under nutrient deficient conditions can have at least about 39%, e.g at least about 39%, 40%, 41%, 42%, 43%, 44%, 45%, 46%, 47%, 48%, 49%, 50%, 51%, 52%, 53%, 54%, 55%, 56%, 57%, 58%, 59%, 60% or more, lipid content (% per ash-free dry weight).

The sugar content of the algal cells or algal biomass can be measured using a phenol sulfuring acid method (Dubois et al., Analytical Chemistry, 28:350-356, 1956), sequential hydrolysis of carbohydrate polymers and identification and quantification of the monomers by high pressure liquid chromatography or gas chromatography (Templeton et al., Journal of Chromatography A, 1270:225-234, 2012).

In some embodiments, the modified algal cell has at least about 50%, e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% less sugar content compared to a wild-type cell under either nutrient sufficient or nutrient deficient conditions.

F. Methods for Extracting Lipids from an Algal Biomass

To generate an algal biomass, standard methods, e.g., flocculation, centrifugation, and filtration (dead end filtration, microfiltration, ultrafiltration, pressure filtration, and tangential flow filtration) can be used for dewatering algae. For instance, cationic chemical flocculants, such as Al₂(SO₄)₃, FeCl₃, and Fe₂(SO₄)₃, can be used to coagulate harvested algae into a biomass.

Algal cells or biomasses can be dried prior to use in obtaining the composition. Standard method of drying an algal biomass include freeze drying, air drying, spray drying, tunnel drying, vacuum drying (lyophilization), and a similar process. Alternatively, a harvested and washed biomass can be used directly produce the composition without drying. In some instances, the biomass is harvested and unwashed prior to performing the extraction method described herein. See, e.g., U.S. Pat. Nos. 5,130,242 and 6,812,009, each of which is herein incorporated by reference in its entirety.

Lipids can be separated from the algal biomass by disruption methods that do not degrade the algal lipids. For instance, the algal cells of the biomass can be disrupted by, e.g., high-pressure homogenization, bead milling, expression/expeller press, sonication, ultrasonication, microwave irradiation, osmotic shock, electromagnetic pulsing, chemical lysis or grinding of dried algal biomass, to release the lipids and other intracellular components. Optionally, the lipids can be separated from the algal cell debris by, e.g., centrifugation. For example, centrifugation produces an oil layer and an aqueous layer containing the cell debris.

Other useful methods tbr extracting lipids from algae include, but are not limited to: Bligh and Dyer's solvent extraction method; solvent extraction with a mixture of ionic liquids and methanol; hexane solvent extraction; ethanol solvent extraction; methanol solvent extraction; soxhlet extraction; supercritical fluid/CO₂ extraction; and organic solvent (e.g., benzene, cyclohexane, hexane, acetone, chloroform) extraction. See, e.g., Ratledge et al. “Chapter 13: Down-Stream Processing, Extraction, and Purification of Single Cell Oils,” Single Cell Oils, ed. Zvi Cohen and Colin Ratledge, AOCS Press, Champaign, Ill., 2005. The extraction method may affect the fatty acid composition recovered from the algal biomass. For instance, the concentration, volume, purity and type of fatty acid may be affected.

The lipids can be further chemically or physically modified or processed by any known technique. For instance, such lipids can be processed to produce various products, such as, but not limited to animal or fish feed, food additives, nutritional products, dietary products, cosmetics, industrial products, and pharmaceutical products.

### IV. EXAMPLES

The following examples are offered to illustrate, but not to limit, the claimed invention.

### Example 1

### Identification of the BGS1 Gene in the Nannochloropsis Oceanica Isolate W2J3B

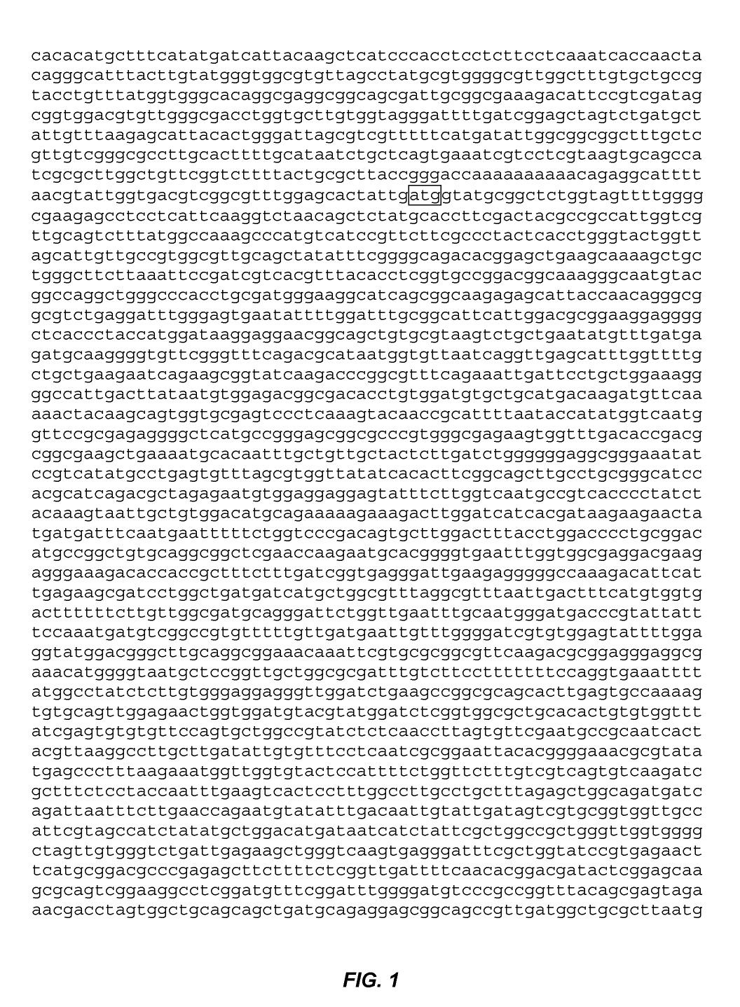

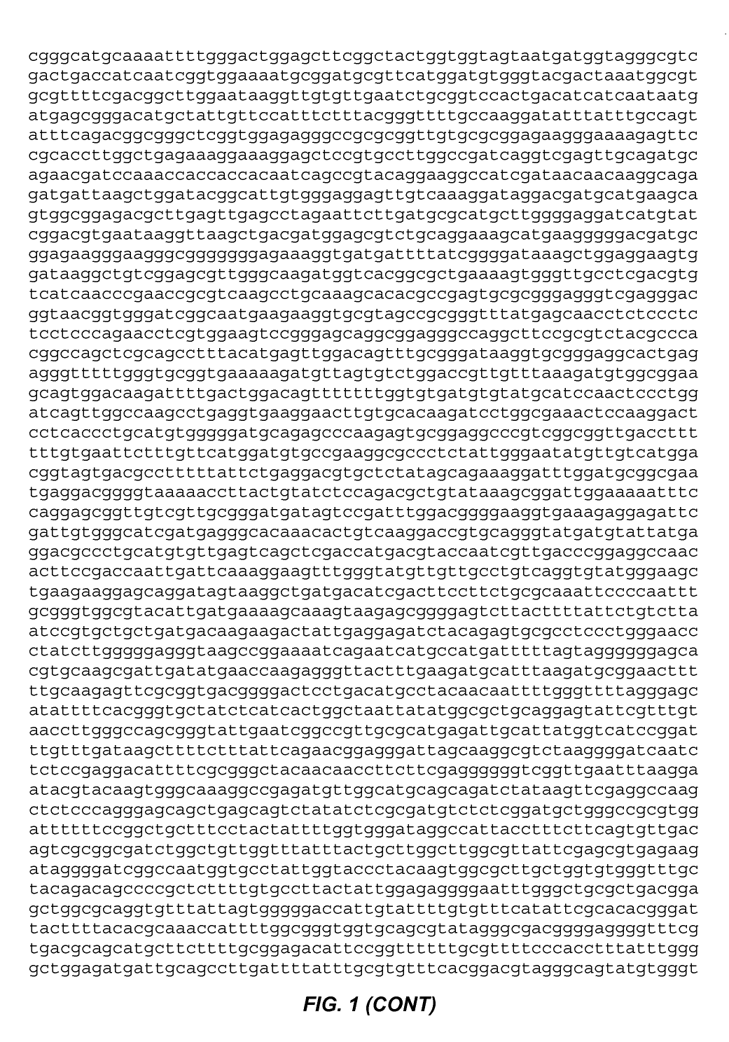

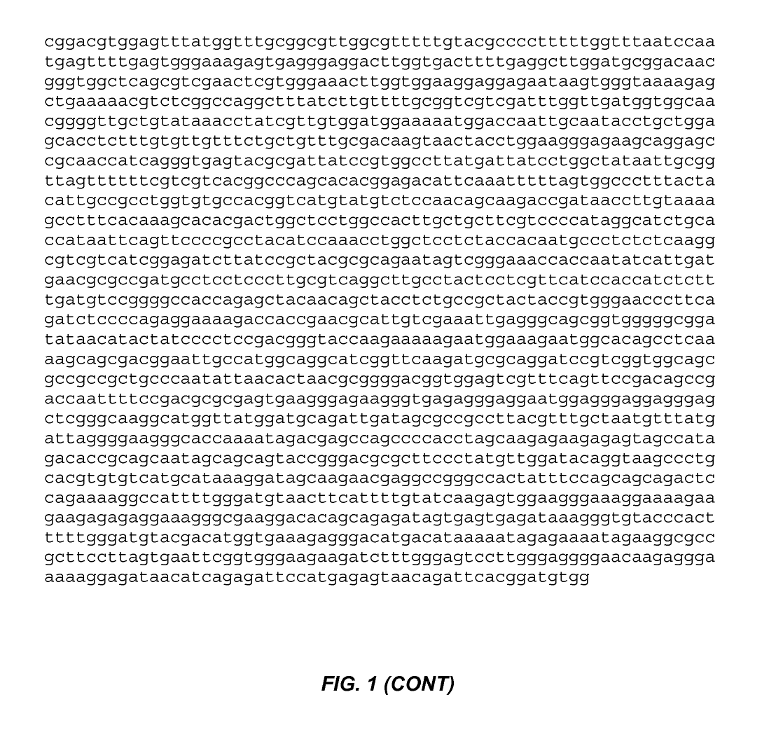

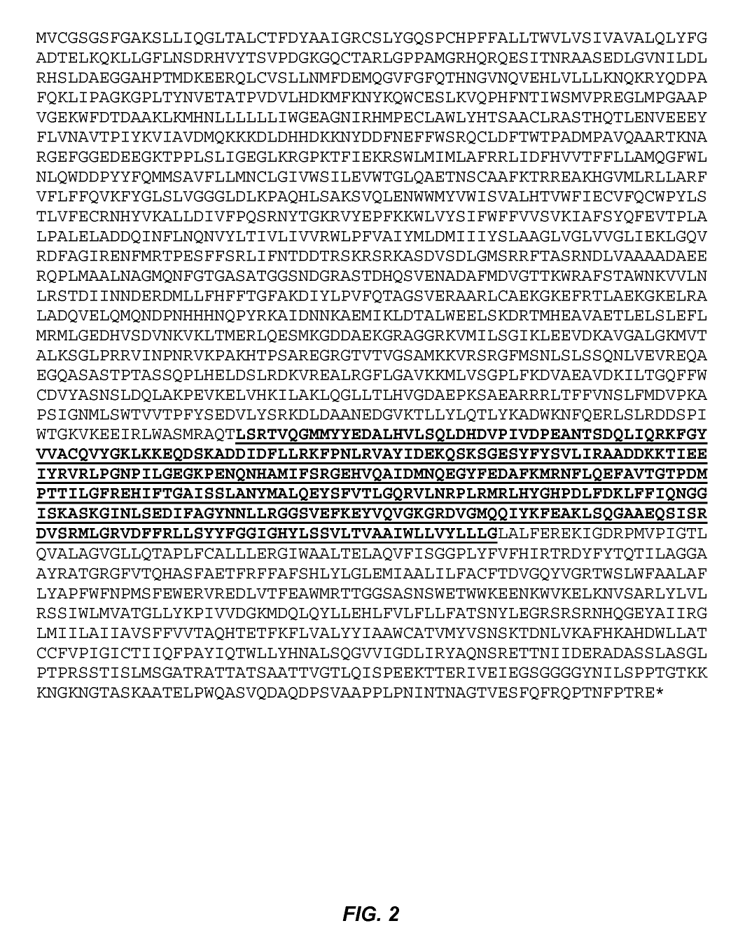

Sequence alignments were performed to identify the beta glucan synthase gene in Nannochloropsis. In particular, tBlastn analysis of the Nannochloropsis W2J3B genome utilizing a callose synthase homologue from Phytophthora infestans (NCBI reference sequence XP_{—}002906408) revealed an open reading frame (ORF) of 6516 bp (FIG. 1) encoding a protein of 2,171 aa (FIG. 2). No introns were identified, as is typical for large ORFs in the Nannochloropsis genome.

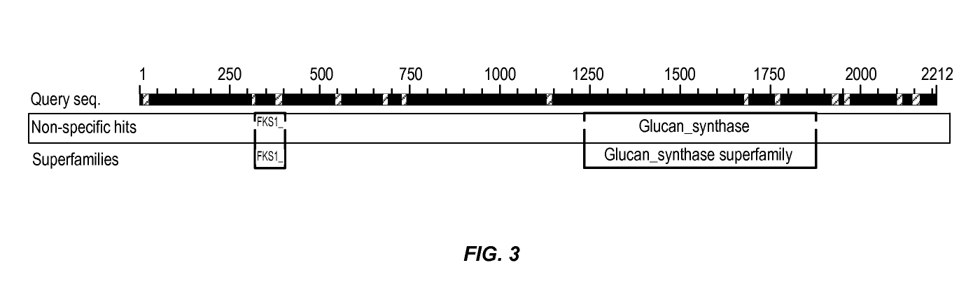

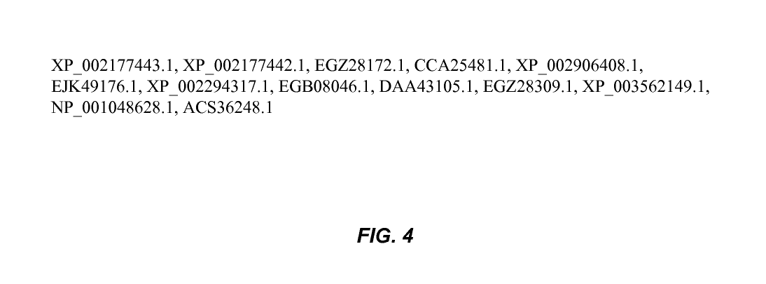

The identified BGS1 protein sequence revealed plain motifs, pfam02364 (FKS-1 domain) and pfam14288 glucan synthase domain). See. FIG. 3. Both Pfam motifs are indicative of 1,3-beta-glucan synthase family members. Homologues to the identified BGS1 sequence include beta glucan synthases in oomycetes and, more importantly, ORFs in other algal species that accumulate polysaccharides composed of beta 1,3-glucan. For instance, predicted BGS1 proteins appear to be present in stramenopiles, such as diatoms and pelagophyceae (e.g., Aureococcus). Examples of BGS1 homologues (FIG. 4) include proteins of NCBI Reference Sequence Nos: XP_{—}002177443.1, XP_{—}002177442.1, XP_{—}002906408.1, XP_{—}002294317.1, XP_{—}003562149.1, and NP_{—}001048628.1; and GenBank Nos. EGZ28172.1, CCA25481.1, EJK49176.1, EGB08046.1, DAA43105.1, EGZ28309.1, and ACS36248.1.

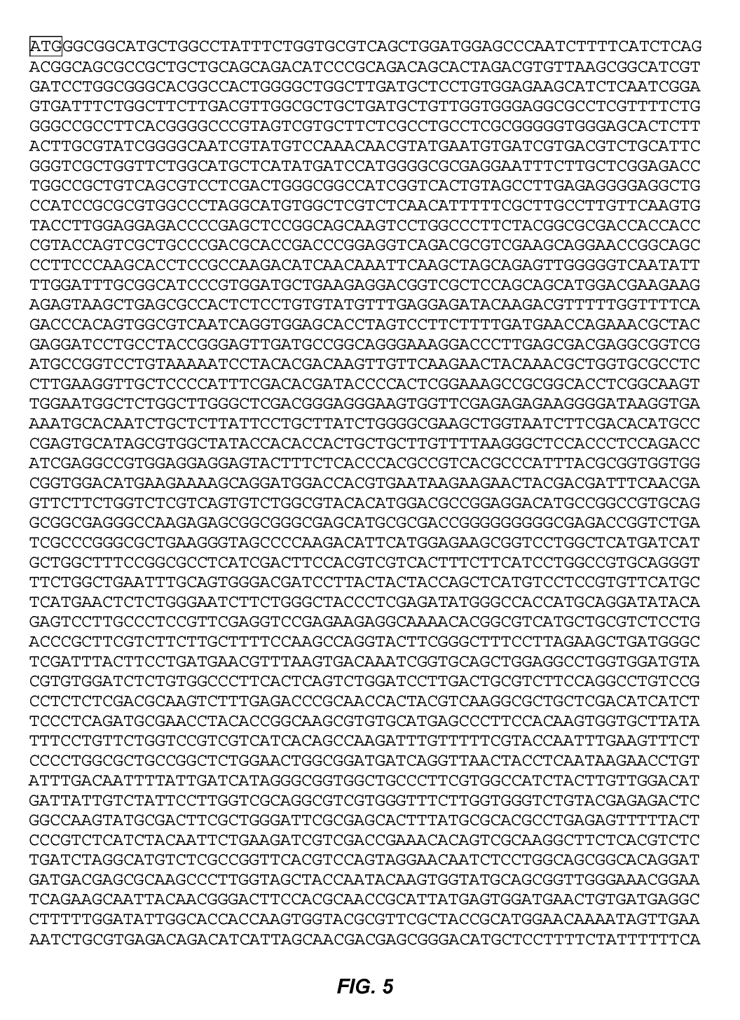

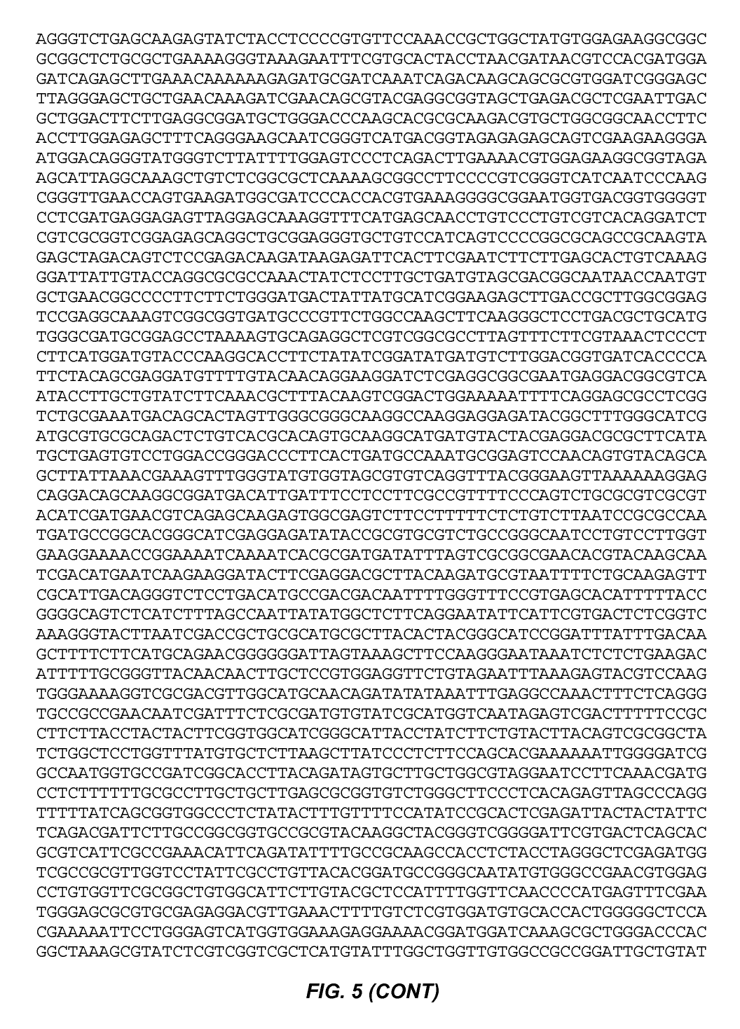

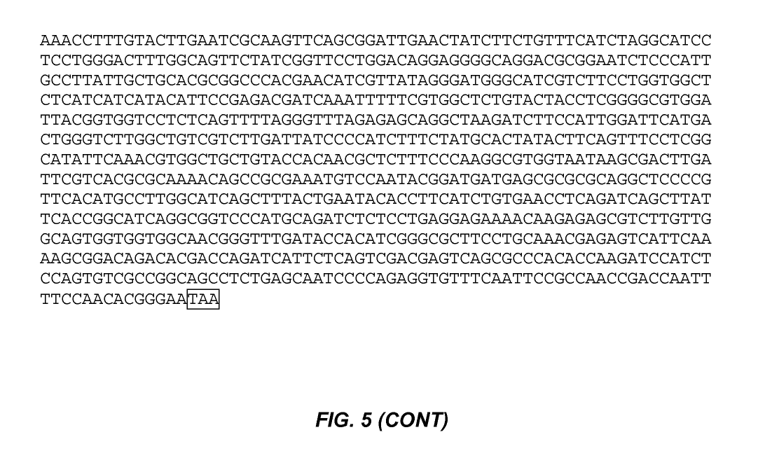

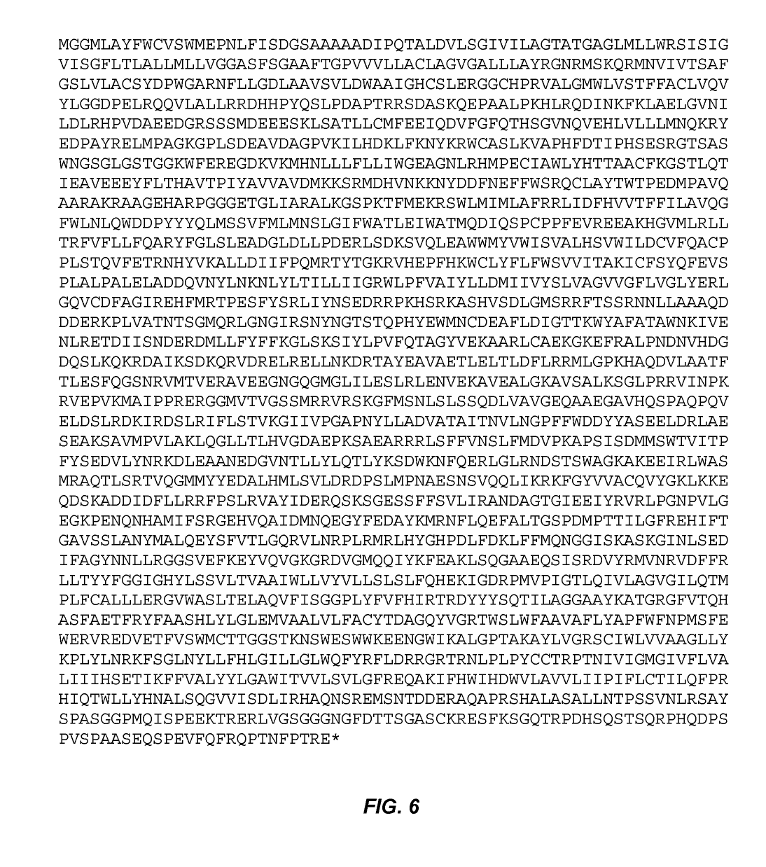

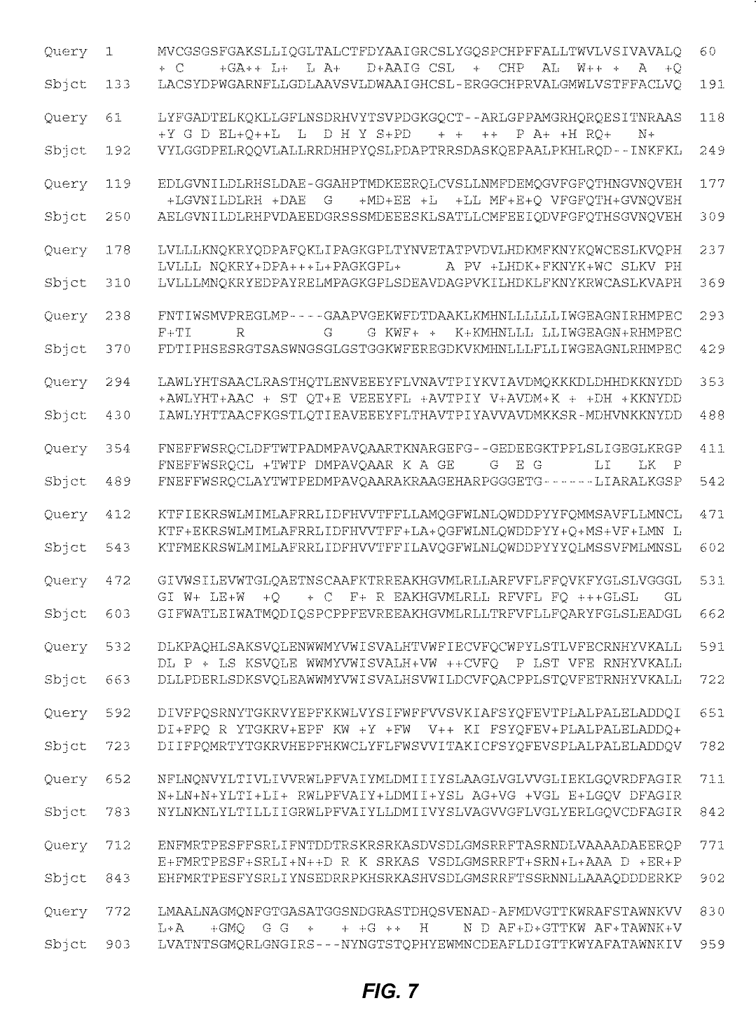

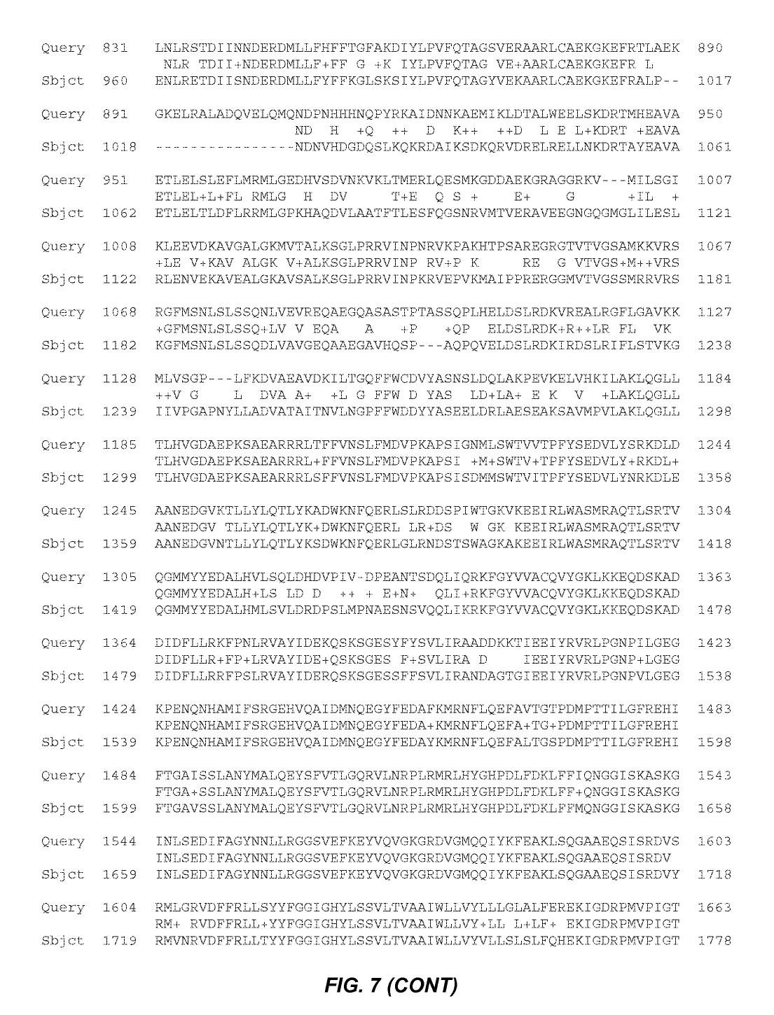

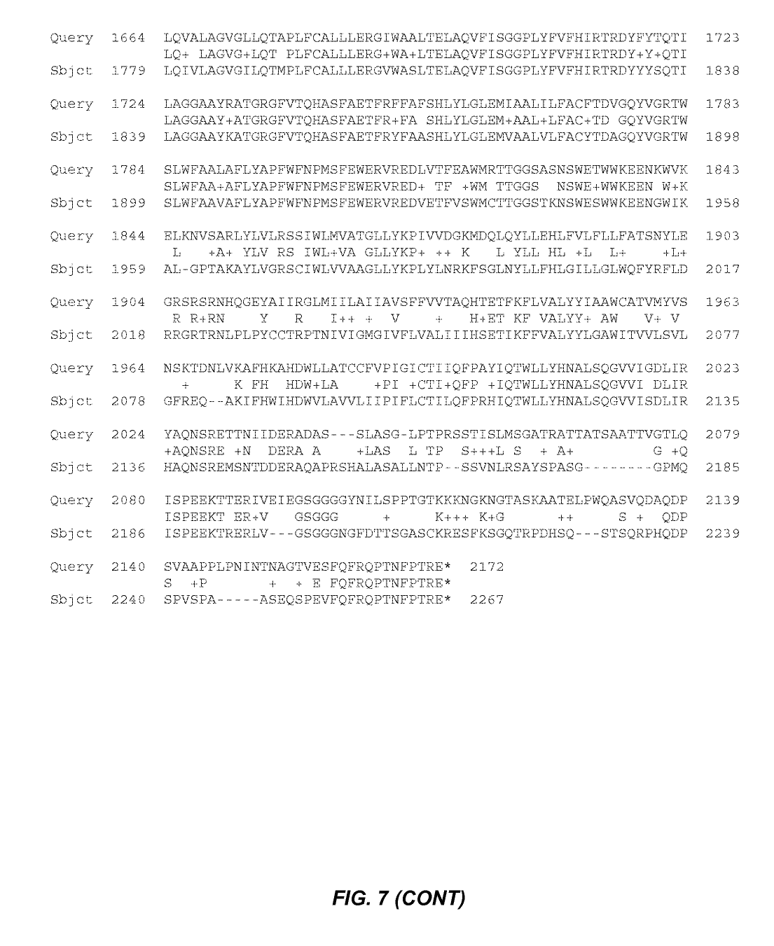

A gene homologue of BGS1 was identified in the Nannochloropsis gaditana isolate IC164 (FIG. 5). The amino acid sequence of the Nannochloropsis gaditana BGS1 protein is shown in FIG. 6. There is 66% sequence identity between the Nannochloropsis oceanica BGS1 protein (“Query”) and the Nannochloropsis gaditana BGS1 (“Sbjct”) (FIG. 7).

### Example 2

### Construction of Targeted Knock-Out Construct for Genomic Disruption of the BGS1 Gene

A knockout (KO) construct for the Nannochloropsis W2J3B BGS1 gene was generated based on the transformation construct NT7 described in U.S. Pat. No. 8,318,482 with the addition of flanking DNA sequences homologous to the BGS1 gene. Detailed descriptions of homologous recombination in algal cells is found in, e.g., Kilian et al., Proc Natl Acad Sci USA, 2011, 108(52):21265-9 and US Patent Publication Nos. 2011/0091977 and 2012/0107801, the contents of which are hereby incorporated by reference in their entirety for all purposes.

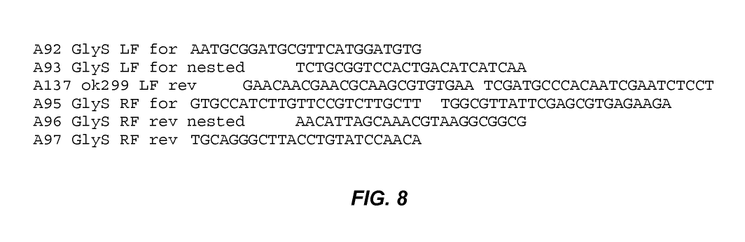

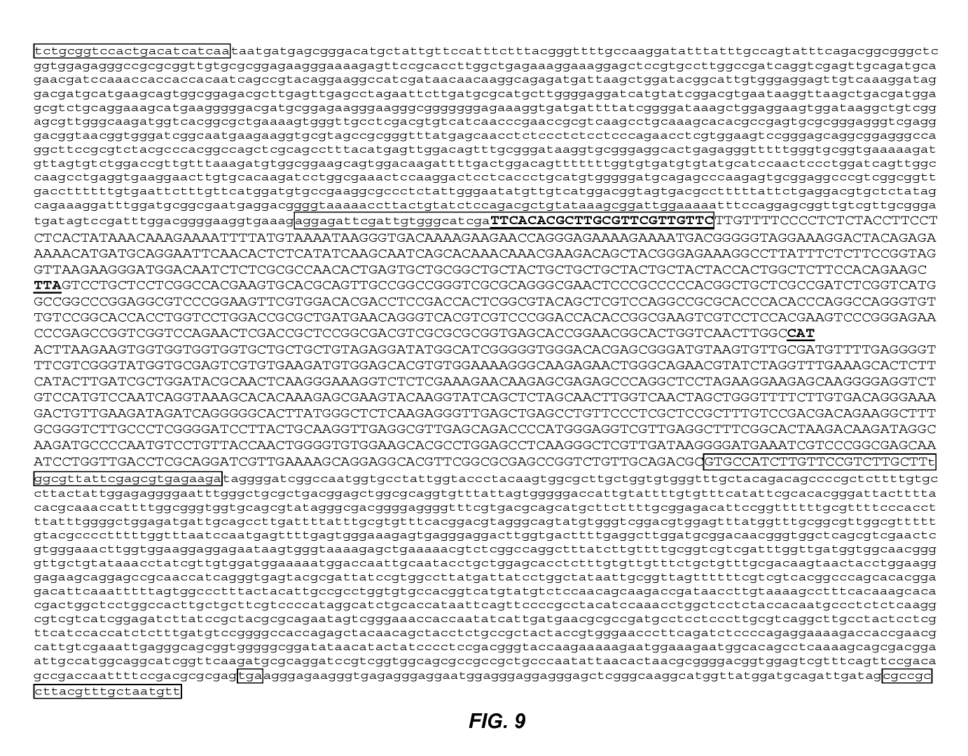

Primers shown in FIG. 8 were used to create the KO construct targeting the BGS1 gene. The left flank was produced by PCR amplification of a BGS1 genomic DNA fragment via primers A92 (SEQ ID NO: 5) and A137 (SEQ ID NO: 7). The right flank was produced by PCR amplification of a BGS1 genomic DNA fragment via primers A95 (SEQ ED NO: 8) and A97 (SEQ ID NO: 10). The flanks were fused to the transformation construct NT7 containing a VCP2 promoter, a sh ble gene and a VCP1 untranslated region by nested PCR utilizing primers A93 (SEQ ID NO: 6) and A96 (SEQ ID NO: 9). See, e.g., U.S. Pat. Nos. 8,318,482 and 8,685,723, and Kilian et al., supra. The nucleotide sequence of the resulting KO construct is depicted in FIG. 9. the final construct included the left flank (1388 bp), a selection marker cassette (1488 bp) and a right flank (1683 bp). Upon homologous recombination, the selection marker cassette is designed to replace the nucleotide sequence encoding amino acids 1299-1644 of the BGS1 protein, which includes most of the BGS1 pfam motif 14288.

### Example 3

### Transformation of the Knock-Out Construct Nannochloropsis W2J3B

The knockout construct depicted in Example 2 was transformed into Nannochloropsis W2J3B as described in Kilian, supra and U.S. Patent Publication Nos. 2011/0091977 and 2012/0107801. Colonies obtained under zeocin selection were screened via PCR for successful KO events.

### Example 4

### Characterization of BGS1 Knock-Out (KO) Mutant (OK299)

The BGS1 KO mutant OK299 was characterized by analyzing lipid content under nitrogen starvation. Cells were grown under constant bubbling of 3% CO₂ enriched air at 200 μmol photons/(m2*s) constant light. Wild-type Nannochloropsis W2J3B and the BGS1 KO mutant OK299 were grown to log phase in nutrient rich medium and subsequently washed in seawater medium without the addition of nutrients, in order to induce starvation conditions. Cells were resuspended in seawater to identical densities and cultures under conditions as described above, with the modification that no nutrients were present. Cultures were grown in biological duplicates.

Samples were taken immediately after washing the cells and from thereon once a day. Samples were analyzed by estimating cell counts, ash-free dry weight, lipid content and sugar content. Lipid content was measured by direct trans-esterification and subsequent gas chromatography analysis. Sugar content was determined according to the methods described in, e.g., Dubois et al., Anal. Chem., 1956, 28:350-356.

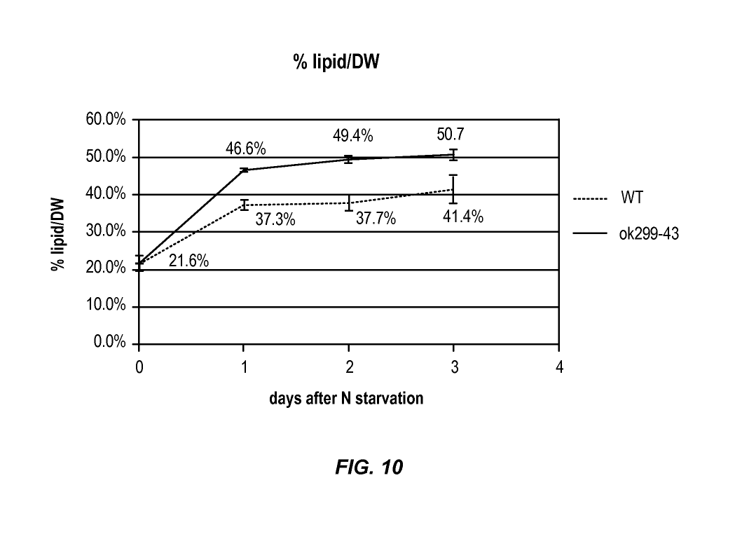

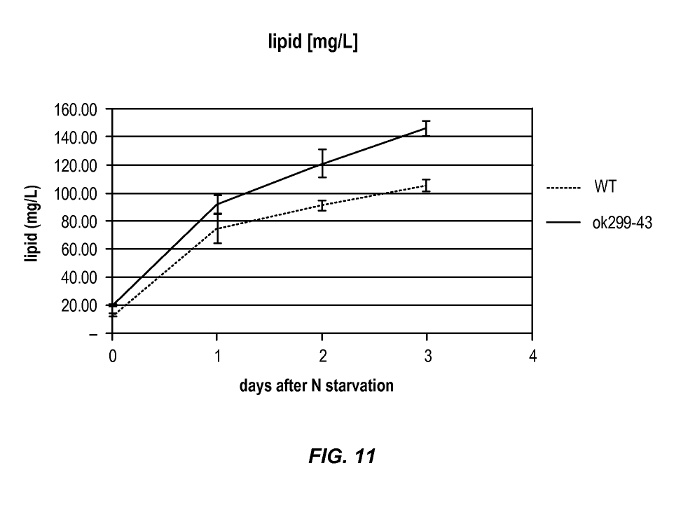

Under nutrient rich conditions, the BGS1 KO mutant OK299 and wild-type had similar lipid content based on ash-free dry weight (AFDW) under nutrient rich conditions (FIG. 10). Yet, under nutrient deficient conditions, the percentage of lipid increased by about 25% in the OK299 cells compared to wildtype on day 1-3 (FIG. 11). In particular, the lipid content per AFDW on day 1 for the BGS1 mutant was 46.6% and 37.3% for the wild-type cells. The BGS1 KO mutant also accumulated more lipid per culture volume (lipid in mg/culture volume; FIG. 11).

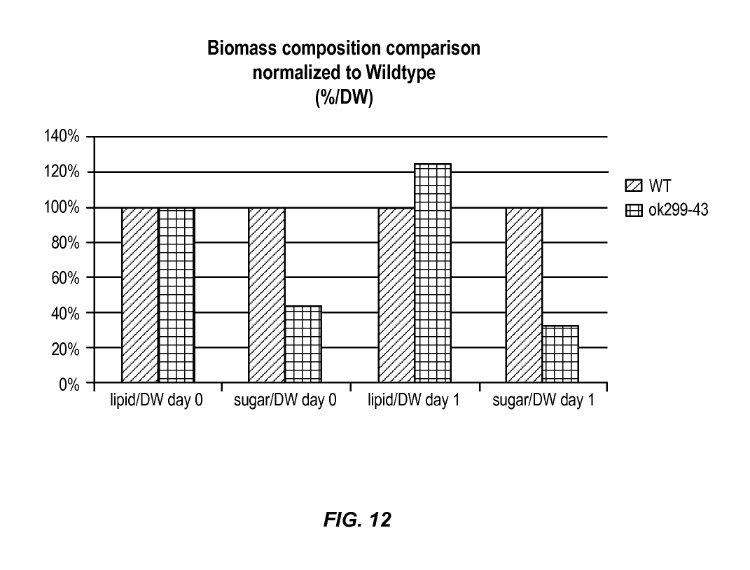

FIG. 12 depicts the amount of sugar and lipid accumulated per AFDW, normalized to wildtype. The BGS1 KO mutant OK299 has similar lipid content as wild-type under nutrient sufficient conditions while lipid content was markedly increased after one day culturing in nutrient deficient conditions (about 25% higher than wildtype). Sugar analysis of these samples revealed that the BGS1 KO mutant had about 60% less glucose equivalents under nutrient sufficient conditions compared to wildtype (FIG. 12). After 1 day of culture under nutrient deficient conditions sugar content further decreased compared to wildtype (about 65% less sugar than wildtype).

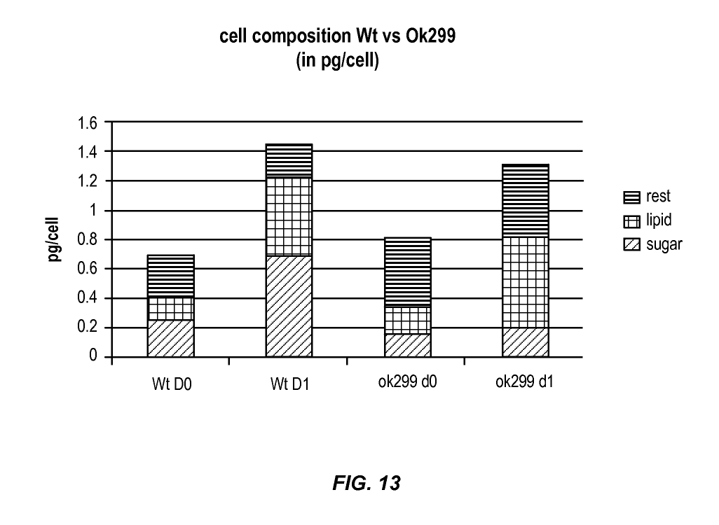

Sugar content per cell was much higher for wild-type than the BGS1 KO mutant under nutrient rich conditions (FIG. 13). This dramatically increased under nutrient deficient condition, indicating active sugar accumulation when wildtype cells are starved for nutrients. Sugar content in the BGS1 KO mutant was low and did not significantly increase during nutrient deficient conditions. These findings indicated that storage sugar accumulation (biosynthesis) was hindered in the BGS1 KO mutant.

In summary, the BGS1 KO mutant accumulated higher amounts of lipids and lower amounts of polysaccharides compared to wild-type. This is likely due to partitioning more carbon flux into the lipid biosynthesis pathway because the polysaccharide biosynthesis is impaired.

Although the foregoing invention has been described in some detail by way of illustration and example for purposes of clarity of understanding, one of skill in the art will appreciate that certain changes and modifications may be practiced within the scope of the appended claims. In addition, each reference cited herein is incorporated by reference in its entirety to the same extent as if each reference was individually incorporated by reference.

### Informal Sequence Listing

```
Beta glucan synthase
Nannochloropsis oceanica W2J3B
SEQ ID NO: 1
      1 cacacatgct ttcatatgat cattacaagc tcatcccacc tcctcttcct caaatcacca
    61 actacagggc atttacttgt atgggtggcg tgttagccta tgcgtggggc gttggctttg
  121 tgctgccgta cctgtttatg gtgggcacag gcgaggcggc agcgattgcg gcgaaagaca
  181 ttccgtcgat agcggtggac gtgttgggcg acctggtgct tgtggtaggg attttgatcg
  241 gagctagtct gatgctattg tttaagagca ttacactggg attagcgtcg tttttcatga
  301 tattggcgac ggctttgctc gttgtcgggc gccttgcact tttgcataat ctgctcagtg
  361 aaatcgtcct cgtaagtgca gccatcgcgc ttggctgttc ggtcttttac tgcgcttacc
  421 gggaccaaaa aaaaaacaga ggcattttaa cgtattggtg acgtcggcgt ttggagcact
  481 attaatggta tgcggctctg gtagttttgg ggcgaagagc ctcctcattc aaggtctaac
  541 agctctatgc accttcgact acgccgccat tggtcgttgc agtctttatg gccaaagccc
  601 atgtcatccg ttcttcgccc tactcacctg ggtactggtt agcattgttg ccgtggcgtt
  661 gcagctatat ttcggggcag acacggagct gaagcaaaag ctgctgggct tcttaaattc
  721 cgatcgtcac gtttacacct cggtgccgga cggcaaaggg caatgtacgg ccaggctggg
  781 cccacctgcg atgggaaggc atcagcggca agagagcatt accaacaggg cggcatctga
  841 ggatttggga gtgaatattt tggatttgcg gcattcattg gacgcggaag gaggggctca
  901 ccctaccatg gataaggagg aacggcagct gtgcgtaagt ctgctgaata tgtttgatga
  961 gatgcaaggg gtgttcgggt ttcagacgca taatggtgtt aatcaggttg agcatttggt
1021 tttgctgctg aagaatcaga agcggtatca agacccggcg tttcagaaat tgattcctgc
1081 tggaaagggg ccattgactt ataatgtgga gacggcgaca cctgtggatg tactgcatga
1141 caagatgttc aaaaactaca agcagtggtg cgagtccctc aaagtacaac cgcattttaa
1201 taccatatgg tcaatggttc cgcgagaggg gctcatgccg ggagcggcgc ccgtgggcga
1261 gaaatggttt gacaccgacg cggcgaagct gaaaatgcac aatttgctgt tactactctt
1321 gatctggggg gaggcgggaa atatccgtca tatgcctgag tgtttagcgt ggttatatca
1381 cacttcggca gcttgcctgc gggcatccac gcatcagacg ctagagaatg tggaggagga
1441 gtatttcttg gtcaatgccg tcacccctat ctacaaagta attgctgtgg acatgcagaa
1501 aaagaaagac ttggatcatc acgataagaa gaactatgat gatttcaatg aatttttctg
1561 atcccgacag tgcttggact ttacctggac ccctgcggac atgccggctg tgcaagcggc
1621 tcgaaccaag aatgcacggg gtgaatttgg tggcgaggac gaagagggaa agacaccacc
1681 gctttctttg atcggtgagg gattgaagag ggggccaaag acattcattg agaagcgatc
1741 ctggctgatg atcatactgg cgtttaggcg tttaattgac tttcatgtgg tgactttttt
1801 cttgttggcg atgcagggat tctggttgaa tttgcaatgg gatgacccgt attatttcca
1861 aatgatgtcg gccgtgtttt tgttgatgaa ttgtttgggg atcgtgtgga gtattttgga
1921 ggtatggacg ggcttgcagg cggaaacaaa ttcgtgcgcg gcgttcaaga cgcggaggga
1981 ggcgaaacat ggggtaatgc tccggttgct ggcgcgattt gtcttccttt ttttccaggt
2041 gaaattttat ggcctatctc ttgtgggagg agggttggat ctgaagccgg cacagcactt
2101 gagtgccaaa agtgtgcagt tggagaactg gtggatgtac gtatggatct cggtggcgct
2161 gcacactgtg tggtttatcg agtgtgtgtt ccagtgctgg ccgtatctct caaccttagt
2221 gttcgaatgc cgcaatcact acattaaggc cttgcttaat attgtgtttc ctcaatcgcg
2281 gaattacacg gggaaacgcg tatatgagcc ctttaagaaa tggttggtgt actccatttt
2341 ctggttcttt gtcgtcagtg tcaagatcgc tttctcctac caatttgaag tcactccttt
2401 ggccttgcct gctttagagc tggcagatga tcagattaat ttcttgaacc agaatgtata
2461 tttgacaatt gtattgatag tcgtgcggtg gttgccattc gtagccatct atatgctgga
2521 catgataatc atctattcgc tggccgctgg gttggtgggg ctagttgtgg gtctaattga
2581 gaagctgggt caagtgaggg atttcgctgg tatccgtgag aacttcatgc ggacgcccga
2641 gagcttcttt tctcggttga ttttcaacac ggacgatact cggagcaagc gcagtcggaa
2701 ggcctcggat gtttcggatt tggggatgtc ccgccggttt acagcgagta gaaacgacct
2761 agtggctgca gcagctgatg cagaggagcg gcagccgttg atggctgcgc ttaatgcggg
2821 catgcaaaat tttgggactg gagcttcggc tactggtggt agtaatgatg gtagggcgtc
2881 gactgaccat caatcggtgg aaaatgcgga tgcgttcatg gatgtgggta cgactaaatg
2941 gcgtgcgttt tcgacggctt ggaataaggt tgtgttgaat ctgcggtcca ctgacatcat
3001 caataatgat gagcgggaca tgctattgtt ccatttcttt acgggttttg ccaaggatat
3061 ttatttgcca gtatttcaga cggcgggctc ggtggagagg gccgcgcggt tgtgcgcgga
3121 gaagggaaaa gagttccgca ccttggctga gaaaggaaag gagctccgtg ccttggccga
3181 tcaggtcgag ttgcagatgc agaacgatcc aaaccaccac cacaatcagc cgtacaggaa
3241 ggccatcgat aacaacaagg cagagatgat taagctggat acggcattgt gggaggagtt
3301 gtcaaaggat aggacgatgc atgaagcagt ggcggagacg cttgagttga gcctagaatt
3361 cttgatgcgc atgcttgggg aggatcatgt atcggacgtg aataaggtta agctgacgat
3421 ggagcgtctg caggaaagca tgaaggggga cgatgcggag aagggaaggg cgggggggag
3481 aaaggtgatg attttatcgg ggataaagct ggaggaagtg gataaggctg tcggagcgtt
3541 gggcaagatg gtcacggcgc tgaaaagtgg gttgcctcga cgtgtcatca acccgaaccg
3601 cgtcaagcct acaaaacaca cgccgagtgc gcgggagggt cgagggacgg taacggtggg
3661 atcggcaatg aagaaggtgc gtagccgcgg gtttatgagc aacctctccc tctcctccca
3721 gaacctcgtg gaagtccggg agcaggcgga gggccaggct tccgcgtcta cgcccacggc
3781 cagctcgcag cctttacatg agttgaacag tttgcgggat aaggtgcggg aggcactgag
3841 agggtttttg ggtgcggtga aaaagatgtt agtgtctgga ccgttgttta aagatgtggc
3901 ggaagcagtg gacaagattt tgactggaca gtttttttgg tgtgatgtgt atgcatccaa
3961 ctccctggat cagttggcca agcctgaggt gaaggaactt gtgcacaaga tcctggcgaa
4021 actccaagga ctcctcaccc tgcatgtggg ggatgcagag cccaagagtg cggaggcccg
4081 tcggcggttg accttttttg tgaattcttt gttcatggat gtgccgaagg caccctctat
4141 tgggaatatg ttgtcatgga cggtagtgac gcctttttat tctgaggacg tgctctatag
4201 cagaaaggat ttggatgcgg cgaatgagga cggggtaaaa accttactgt atctccagac
4261 gctgtataaa gcggattgga aaaatttcca ggagcggttg tcgttgcggg atgatagtcc
4321 gatttggacg gggaaggtga aagaggagat tcgattgtgg gcatcgatga gggcacaaac
4381 actgtcaagg accgtacagg gtatgatgta ttatgaggac gccctgcatg tgttaagtca
4441 gctcgaccat gacgtaccaa tcgttgaccc ggaggccaac acttccgacc aattgattca
4501 aaggaagttt gggtatgttg ttgcctgtca ggtgtatggg aagctgaaga aggagcagga
4561 tagtaaggct gatgacatcg acttccttct gcgcaaattc cccaatttgc gggtggcgta
4621 cattgatgaa aagcaaagta agagcgggga gtcttacttt tattctgtct taatccgtgc
4681 tgctgatgac aagaagacta ttgaggagat ctacagagtg cgcctccctg ggaaccctat
4741 cttgggggag ggtaagccgg aaaatcagaa tcatgccatg atttttagta ggggggagca
4801 cgtgcaagcg attgatatga accaagaggg ttactttgaa gatgcattta agatgcggaa
4861 ctttttgcaa gagttcgcgg tgacggggac tcctgacatg cctacaacaa ttttgggttt
4921 tagggagcat attttcacgg gtgctatctc atcactggct aattatatgg cgctgcagga
4981 gtattcgttt gtaaccttgg gccagcgggt attgaatcgg ccgttgcgca tgagattgca
5041 ttatggtcat ccggatttat ttgataagct tttctttatt cagaacggag ggattagcaa
5101 ggcgtctaag gggatcaatc tctccgagga cattttcgcg ggctacaaca accttcttcg
5161 aggggggtcg gttgaattta aggaatacgt acaagtgggc aaaggccgag atgttggcat
5221 gcagcagatc tataagttcg aggccaagct ctcccaggga gcagctgagc agtctatatc
5281 tcgcgatgtc tctcggatgc tgggccgcgt ggattttttc cggctgcttt cctactattt
5341 tggtgggata ggccattacc tttcttcagt gttgacagtc gcggcgatct ggctattggt
5401 ttatttactg cttggcttgg cgttattcga gcgtgagaag ataggggatc ggccaatggt
5461 gcctattggt accctacaag tggcgcttgc tggtgtgggt ttgctacaga cagccccgct
5521 cttttgtgcc ttactattgg agaggagaat ttgggctgcg ctgacggagc tggcacaggt
5581 gtttattagt gggggaccat tgtattttgt gtttcatatt cgcacacggg attactttta
5641 cacgcaaacc attttggcgg gtggtgcagc gtatagggcg acggggaggg gtttcgtgac
5701 gcagcatgct tcttttgcgg agacattccg gttttttgcg ttttcccacc tttatttggg
5761 gctggagatg attgcagcct tgattttatt tgcgtgtttc acggacgtag ggcagtatgt
5821 gggtcggacg tggagtttat ggtttgcggc gttggcgttt ttgtacgccc ctttttggtt
5881 taatccaatg agttttgagt gggaaagagt gagggaggac ttggtgactt ttgaggcttg
5941 gatgcggaca acgggtggct cagcgtcgaa ctcgtgggaa acttggtgga aggaggagaa
6001 taagtgggta aaagagctga aaaacgtctc ggccaggctt tatcttgttt tgcggtcgtc
6061 gatttggttg atggtggcaa cggggttgct gtataaacct atcgttgtgg atggaaaaat
6121 ggaccaattg caatacctgc tggagcacct ctttgtgttg tttctgctgt ttgcgacaag
6181 taactacctg gaagggagaa gcaggagccg caaccatcag ggtgagtacg cgattatccg
6241 tggccttatg attatcctgg ctataattgc ggttagtttt ttcgtcgtca cggcccagca
6301 cacggagaca ttcaaatttt tagtggccct ttactacatt gccgcctggt gtgccacggt
6361 catgtatgtc tccaacagca agaccgataa ccttgtaaaa gcctttcaca aagcacacga
6421 ctggctcctg gccacttgct gcttcgtccc cataggcatc tgcaccataa ttcagttccc
6481 cgcctacatc caaacctggc tcctctacca caatgccctc tctcaaggcg tcgtcatcgg
6541 agatcttatc cgctacgcgc agaatagtcg ggaaaccacc aatatcattg atgaacgcgc
6601 cgatgcctcc tcccttgcgt caggcttgcc tactcctcgt tcatccacca tctctttgat
6661 gtccggggcc accagagcta caacagctac ctctgccgct actaccgtgg gaacccttca
6721 gatctcccca gaggaaaaga ccaccgaacg cattgtcgaa attgagggca gcggtggggg
6781 cggatataac atactatccc ctccgacggg taccaagaaa aagaatgaaa aaaatggcac
6841 agcctcaaaa gcagcgacgg aattgccatg gcaggcatcg gttcaagatg cgcaggatcc
6901 gtcggtggca gcgccgccgc tgcccaatat taacactaac gcggggacgg tggagtcgtt
6961 tcagttccga cagccgacca attttccgac gcgcgagtga agggagaagg gtgagaggga
7021 ggaatggagg gaggagggag ctcgggcaag gcatggttat ggatgcagat tgatagcgcc
7081 accttacgtt tgctaatgtt tatgattagg ggaagggcac caaaatagac gagccagccc
7141 cacctagcaa gagaagagag tagccataga caccgcagca atagcagcag taccgggacg
7201 cgcttcccta tgttggatac aggtaagccc tgcacgtgtg tcatgcataa aggatagcaa
7261 gaacgaggcc gggccactat ttccagcagc agactccaga aaaggccatt ttgggatgta
7321 acttcatttt gtatcaagag tggaagggaa aggaaaagaa gaagagagag gaaagggcga
7381 aggacacagc agagatagtg agtgaaataa agggtgtacc cactttttgg gatgtacgac
7441 atggtgaaag agggacatga cataaaaata gagaaaatag aaggcgccgc ttccttagtg
7501 aattcggtgg gaagaagatc tttgggagtc cttgggaggg gaacaagagg gaaaaaggag
7561 ataacatcag agattccatg agagtaacag attcacggat gtgg
Beta glucan synthase protein
Nannochloropsis W2J3R
SEQ ID NO: 2
      1 MVCGSGSFGA KSLLIQGLTA LCTFDYAATG RCSLYGQSPC RPFFALLTWV LVSIVAVALQ
    61 LYFGADTELK QKLLGFLNSD RHVYTSVPDG KGQCTARLGP PAMGRHQRQE SITNRAASED
  121 LGVNILDLRH SLDAEGGARP TMDEEERQLC VSLLNMFDFM QGVFGFQTHN GVNQVEHLVL
  181 LLKNQKRYQD PAFQKLIPAG KGPLTYNVET ATPVDVLHDK MFKNYKQWCE SLKVQPHFNT
  241 IWSMVPREGL MPGAAPVGEK WFDTDAAKLK MHNLLLLLLI WGEAGNIRHM PECLAWLYHT
  301 SAACLRASTH QTLENVEEEY FLVNAVTPIY KVIAVDMQRK KDLDHHDKKN YDDFNEFFWS
  361 RQCLDFTWTP ADMPAVQAAR TKNARGEFGG EDEEGKTPPL SLIGEGLKRG PKTFIEKRSW
  421 LMIMLAFRRL IDFHVVTFFL LAMQGFWLNL QWDDPYYFQM MSAVFLLMNC LGIVWSILEV
  481 WTGLQAETNS CAAFKTRREA KHGVMLRLLA RFVFLFFQVK FYGLSLVGGG LDLKPAQHLS
  541 AKSVQLENWW MYVWISVALH TVWFIECVFQ CWPYLSTLVF ECRNHYVKAL LDIVFPQSRN
  601 YTBKRVTEPF KKWLVYSIFW FFVVSVKIAF SYQFEVTPLA LPALELADDQ INFLNQNVYL
  661 TIVLIVVRWL PFVAIYMLDM IIIYSLAAGL VGLVVGLIEK LGQVRDFAGI RENFMRTPES
  721 FFSPLIFNTD DTRSKRSRKA SDVSDLGMSR RFTASRNDLV AAAADAEERQ PLMAALNQGM
  781 QNFGTGASAT GGSNDGRAST DHQSVENADA FMDVGTTKWR AFSTAWNKVV LNLRSTDIIN
  841 NDERDMLLFH FFTGFAKDIY LPVFQTAGSV ERAARLCAEK GKEFRTLAEK GKELRALADQ
  901 VELQMQNDPN HHHNQPYRKA IDNNKAEMIK LDTALWEELS KDRTMHEAVA ETLELSLEFL
  961 MPMLGEDHVS DVNKVKLTME PLQESMKGDD AEKGRAGGRK VMILSGIKLE EVDKAVGALG
1021 KMVTALKSGL PRRVINPNRV KPAKHTPSAR EGRGTVTVGS AMKKVRSRGF MSNLSLSSQN
1081 LVEVREQAEG QASASTPTAS SQPLHELDSL RDKVREALRG FLGAVKKMLV SGPLFKDVAE
1141 AVDKILTGQF FWCDVYASNS LDQLAKPEVK ELVHKILAKL QGLLTLHVGD AEPKSAEARP
1201 RLTFFVNSLF MDVPKAPSIG NMLSWTVVTP FYSEDVLYSP KDLDAANEDG VKTLLYLQTL

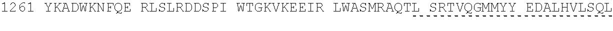

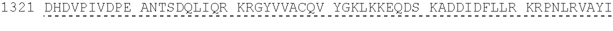

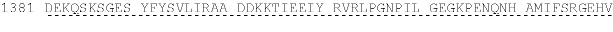

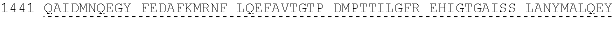

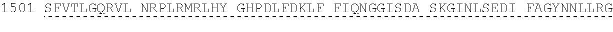

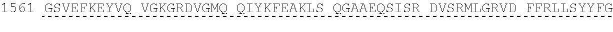

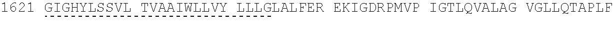







1681 CALLLERGIW AALTELAQVF ISGGPLYFVF HIRTRDYFYT QTILAGGAAY RATGRGFVTQ
1741 HASFAETFRF FAFSHLYLGL EMIAALILFA CFTDVGQYVG RTWSLWFAAL AFLYAPFWFN
1801 PMSFEWERVR EDLVTFEAWM RTTGGSASNS WETWWKEENK WVKELKNVSA RLYLVLRSSI
1861 WLMVATGLLY KPIVVDGKMD QLQYLLEHLF VLFLLFATSN YLEGRSRSRN HQGEYAIIRG
1921 LMIILAIIAV SFFVVTAQHT ETFKFLVALY YIAAWCATVM YVSNSKTDNL VKAFHKAHDW
1981 LLATCCFVPI GICTIIQFPA YIQTWLLYHN ALSQGVVIGD LIRYAQNSRE TTNIIDERAD
2041 ASSLASGLPT PRSSTISLMS GATRATTATS AATTVGTLQI SPEEKTTERI VEIEGSGGGG
2101 YNILSPPTGT KKKNGKNGTA SKAATELPWQ ASVQDAQDPS VAAPPLPNIN TNAGTVESFQ
2161 FRQPTNFPTR E*
Beta caucan synthase
Nannochloropsis gaditana IC164 isolate
SEQ ID NO: 3
      1 ATGGGCGGCA TGCTGGCCTA TTTCTGGTGC GTCAGCTGGA TGGAGCCCAA TCTTTTCATC
    61 TCAGACGGCA GCGCCGCTGC TGCAGCAGAC ATCCCGCAGA CAGCACTAGA CGTGTTAAGC
  121 GGCATCGTGA TCCTGGCGGG CACGGCCACT GGGGCTGGCT TGATGCTCCT GTGGAGAAGC
  181 ATCTCAATCG GAGTGATTTC TGGCTTCTTG ACGTTGGCGC TGCTGATGCT GTTGGTGGGA
  241 GGCGCCTCGT TTTCTGGGGC CGCCTTCACG GGGCCCGTAG TCGTGCTTCT CGCCTGCCTC
  301 GCGGGGGTGG GAGCACTCTT ACTTGCGTAT CGGGGCAATC GTATGTCCAA ACAACGTATG
  361 AATGTGATCG TGACGTCTGC ATTCGGGTCG CTGGTTCTGG CATGCTCATA TGATCCATGG
  421 GGCGCGAGGA ATTTCTTGCT CGGAGACCTG GCCGCTGTCA GCGTCCTCGA CTGGGCGGCC
  481 ATCGGTCACT GTAGCCTTGA GAGGGGAGGC TGCCATCCGC GCGTGGCCCT AGGCATGTGG
  541 CTCGTCTCAA CATTTTTCGC TTGCCTTGTT CAAGTGTACC TTGGAGGAGA CCCCGAGCTC
  601 CGGCAGCAAG TCCTGGCCCT TCTACGGCGC GACCACCACC CGTACCAGTC GCTGCCCGAC
  661 GCACCGACCC GGAGGTCAGA CGCGTCGAAG CAGGAACCGG CAGCCCTTCC CAAGCACCTC
  721 CGCCAAGACA TCAACAAATT CAAGCTAGCA GAGTTGGGGG TCAATATTTT GGATTTGCGG
  781 CATCCCGTGG ATGCTGAAGA GGACGGTCGC TCCAGCAGCA TGGACGAAGA AGAGAGTAAG
  841 CTGAGCGCCA CTCTCCTGTG TATGTTTGAG GAGATACAAG ACGTTTTTGG TTTTCAGACC
  901 CACAGTGGCG TCAATCAGGT GGAGCACCTA GTCCTTCTTT TGATGAACCA GAAACGCTAC
  961 GAGGATCCTG CCTACCGGGA GTCGATGCCG GCAGGGAAAG GACCCTTGAG CGACGAGGCG
1021 GTCGATGCCG GTCCTGTAAA AATCCTACAC GACAAGTTGT TCAAGAACTA CAAACGCTGG
1081 TGCGCCTCCT TGAAGGTTGC TCCCCATTTC GACACGATAC CCCACTCGGA AAGCCGCGGC
1141 ACCTCGGCAA GTTGGAATGG CTCTGGCTTG GGCTCGACGG GAGGGAAGTG GTTCGAGAGA
1201 GAAGGGGATA AGGTGAAAAT GCACAATCTG CTCTTATTCC TGCTTATCTG GGGCGAAGCT
1261 GGTAATCTTC GACACATGCC CGAGTGCATA GCGTGGCTAT ACCACACCAC TGCTGCTTGT
1321 TTTAAGGGCT CCACCCTCCA GACCATCGAG GCCGTGGAGG AGGAGTACTT TCTCACCCAC
1381 GCCGTCACGC CCATTTACGC GGTGGTGGCG GTGGACATGA AGAAAAGCAG GATGGACCAC
1441 GTGAATAAGA AGAACTACGA CGATTTCAAC GAGTTCTTCT GGTCTCGTCA GTGTCTGGCG
1501 TACACATGGA CGCCGGAGGA CATGCCGGCC GTGCAGGCGG CGAGGGCCAA GAGAGCGGCG
1561 GGCGAGCATG CGCGACCGGG GGGGGGCGAG ACCGGTCTGA TCGCCCGGGC GCTGAAGCGT
1621 AGCCCCAAGA CATTCATGGA GAAGCGGTCC TGGCTCATGA TCATGCTGGC TTTCCGGCGC
1681 CTCATCGACT TCCACGTCGT CACTTTCTTC ATCCTGGCCG TGCAGGGTTT CTGGCTGAAT
1741 TTGCAGTGGG ACGATCCTTA CTACTACCAG CTCATGTCCT CCCGTCTCAT GCTCATGAAC
1801 TCTCTGGGAA TCTTCTGGGC TACCCTCGAG ATATGGGCCA CCATGCAGGA TATACAGAGT
1861 CCTTGCCCTC CGTTCGAGGT CCGAGAAGAG GCAAAACACG GCGTCATGCT GCGTCTCCTG
1921 ACCCGCTTCG TCTTCTTGCT TTTCCAAGCC AGGTACTTCG GGCTTTCCTT AGAAGCTGAT
1981 GGGCTCGATT TACTTCCTGA TGAACGTTTA AGTGACAAAT CGGTGCAGCT GGAGGCCTGG
2041 TGGATGTACG TGTGGATCTC TGTGGCCCTT CACTCAGTCT GGATCCTTGA CTGCGTCTTC
2101 CAGGCCTGTC CGCCTCTCTC GACGCAAGTC TTTGAGACCC GCAACCACTA CGTCAAGGCG
2161 CTGCTCGACA TCATCTTCCC TCAGATGCGA ACCTACACCG GCAAGCGTGT GCATGAGCCC
2221 TTCCACAAGT GGTGCTTATA TTTCCTGTTC TGGTCCGTCG TCATCACAGC CAAGATTTGT
2281 TTTTCGTACC AATTTGAAGT TTCTCCCCTG GCGCTGCCGG CTCTGGAACT GGCGGATGAT
2341 CAGGTTAACT ACCTCAATAA GAACCTGTAT TTGACAATTT TATTGATCAT AGGGCGGTGG
2401 CTGCCCTTCG TGGCCATCTA CTTGTTGGAC ATGATTATTG TCTATTCCTT GGTCGCAGGC
2461 GTCGTGGGTT TCTTGGTGGG TCTGTACGAG AGACTCGGCC AAGTATGCGA CTTCGCTGGG
2521 ATTCGCGAGC ACTTTATGCG CACGCCTGAG AGTTTTTACT CCCGTCTCAT CTACAATTCT
2581 GAAGATCGTC GACCGAAACA CAGTCGCAAG GCGTCTCCTG TCTCTGATCT AGGCATGTCT
2641 CGCCGGTTCA CGTCCAGTAG GAACAATCTC CTGGCAGCGG CACAGGATGA TGACGAGCGC
2701 AAGCCCTTGG TAGCTACCAA TACAAGTGGT ATGCAGCGGT TGGGAAACGG AATCAGAAGC
2761 AATTACAACG GGACTTCCAC GCAACCGCAT TATGAGTGGA TGAACTGTGA TGAGGCCTTT
2821 TTGGATATTG GCACCACCAA GTGGTACGCG TTCGCTACCG CATGGAACAA AATAGTTGAA
2881 AATCTGCGTG AGACAGACAT CATTAGCAAC GACGAGCGGG ACATGCTCCT TTTCTATTTT
2941 TTCAAGGGTC TGAGCAAGAG TATCTACCTC CCCGTGTTCC AAACCGCTGG CTATGTGGAG
3001 AAGGCGGCGC GGCTCTGCGC TGAAAAGGGT AAAGAATTTC GTGCACTACC TAACGATAAC
3061 GTCCACGATG GAGATCAGAG CTTGAAACAA AAAAGAGATG CGATCAAATC AGACAAGCAG
3121 CGCGTGGATC GGGAGCTTAG GGAGCTGCTG AACAAAGATC GAACAGCGTA CGAGGCGGTA
3181 GCTGAGACGC TCGAATTGAC GCTGGACTTC TTGAGGCGGA TGCTGGGACC CAAGCACGCG
3241 CAAGACGTGC TGGCGGCAAC CTTCACCTTG GAGAGCTTTC AGGGAAGCAA TCGGGTCATG
3301 ACGGTAGAGA GAGCAGTCGA AGAAGGGAAT GGACAGGGTA TGGGTCTTAT TTTGGAGTCC
3361 CTCAGACTTG AAAACGTGGA GAAGGCGGTA GAAGCATTAG GCAAAGCTGT CTCGGCGCTC
3421 AAAAGCGGCC TTCCCCGTCG GGTCATCAAT CCCAAGCGGG TTGAACCAGT GAAGATGGCG
3481 ATCCCACCAC GTGAAAGGGG CGGAATGGTG ACGGTGGGGT CCTCGATGAG GAGAGTTAGG
3541 AGCAAAGGTT TCATGAGCAA CCTGTCCCTG TCGTCACAGG ATCTCGTCGC GGTCGGAGAG
3601 CAGGCTGCGG AGGGTGCTGT CCATCAGTCC CCGGCGCAGC CGCAAGTAGA GCTAGACAGT
3661 CTCCGAGACA AGATAAGAGA TTCACTTCGA ATCTTCTTGA GCACTGTCAA AGGGATTATT
3721 GTACCAGGCG CGCCAAACTA TCTCCTTGCT GATGTAGCGA CGGCAATAAC CAATGTGCTG
3781 AACGGCCCCT TCTTCTGGGA TGACTATTAT GCATCGGAAG AGCTTGACCG CTTGGCGGAG
3841 TCCGAGGCAA AGTCGGCGGT GATGCCCGTT CTGGCCAAGC TTCAAGGGCT CCTGACGCTG
3901 CATGTGGGCG ATGCGGAGCC TAAAAGTGCA GAGGCTCGTC GGCGCCTTAG TTTCTTCGTA
3961 AACTCCCTCT TCATGGATGT ACCCAAGGCA CCTTCTATAT CGGATATGAT GTCTTGGACG
4021 GTGATCACCC CATTCTACAG CGAGGATGTT TTGTACAACA GGAAGGATCT CGAGGCGGCG
4081 AATGAGGACG GCGTCAATAC CTTGCTGTAT CTTCAAACGC TTTACAAGTC GGACTGGAAA
4141 AATTTTCAGG AGCGCCTCGG TCTGCGAAAT GACAGCACTA GTTGGGCGGG CAAGGCCAAG
4201 GAGGAGATAC GGCTTTGGGC ATCGATGCGT GCGCAGACTC TGTCACGCAC AGTGCAAGGC
4261 ATGATGTACT ACGAGGACGC GCTTCATATG CTGAGTGTCC TGGACCGGGA CCCTTCACTG
4321 ATGCCAAATG CGGAGTCCAA CAGTGTACAG CAGCTTATTA AACGAAAGTT TGGGTATGTG
4381 GTAGCGTGTC AGGTTTACGG GAAGTTAAAA AAGGAGCAGG ACAGCAAGGC GGATGACATT
4441 GATTTCCTCC TTCGCCGTTT TCCCAGTCTG CGCGTCGCGT ACATCGATGA ACGTCAGAGC
4501 AAGAGTGGCG AGTCTTCCTT TTTCTCTGTC TTAATCCGCG CCAATGATGC CGGCACGGGC
4561 ATCGAGGAGA TATACCGCGT GCGTCTGCCG GGCAATCCTG TCCTTGGTGA AGGAAAACCG
4621 GAAAATCAAA ATCACGCGAT GATATTTAGT CGCGGCGAAC ACGTACAAGC AATCGACATG
4681 AATCAAGAAG GATACTTCGA GGACGCTTAC AAGATGCGTA ATTTTCTGCA AGAGTTCGCA
4741 TTGACAGGGT CTCCTGACAT GCCGACGACA ATTTTGGGTT TCCGTGAGCA CATTTTTACC
4801 GGGGCAGTCT CATCTTTAGC CAATTATATG GCTCTTCAGG AATATTCATT CGTGACTCTC
4861 GGTCAAAGGG TACTTAATCG ACCGCTGCGC ATGCGCTTAC ACTACGGGCA TCCGGATTTA
4921 TTTGACAAGC TTTTCTTCAT GCAGAACGGG GGGATTAGTA AAGCTTCCAA GGGAATAAAT
4981 CTCTCTGAAG ACATTTTTGC GGGTTACAAC AACTTGCTCC GTGGAGGTTC TGTAGAATTT
5041 AAAGAGTACG TCCAAGTGGG AAAAGGTCGC GACGTTGGCA TGCAACAGAT ATATAAATTT
5101 GAGGCCAAAC TTTCTCAGGG TGCCGCCGAA CAATCGATTT CTCGCGATGT GTATCGCATG
5161 GTCAATAGAG TCGACTTTTT CCGCCTTCTT ACCTAGTACT TCGGTGGCAT CGGGCATTAC
5221 CTATCTTCTG TACTTACAGT CGCGGCTATC TGGCTCCTGG TTTATGTGCT CTTAAGCTTA
5281 TCCCTCTTCC AGCACGAAAA AATTGGGGAT CGGCCAATGG TGCCGATCGG CACCTTACAG
5341 ATAGTGCTTG CTGGCGTAGG AATCCTTCAA ACGATGCCTC TTTTTTGCGC CTTGCTGCTT
5401 GAGCGCGGTG TCTGGGCTTC CCTCACAGAG TTAGCCCAGG TTTTTATCAG CGGTGGCCCT
5461 CTATACTTTG TTTTCCATAT CCGCACTCGA GATTACTACT ATTCTCAGAC GATTCTTGCC
5521 GGCGGTGCCG CGTACAAGGC TACGGGTCGG GGATTCGTGA CTCAGCACGC GTCATTCGCC
5581 GAAACATTCA GATATTTTGC CGCAAGCCAC CTCTACCTAG GGCTCGAGAT GGTCGCCGCG
5641 TTGGTCCTAT TCGCCTGTTA CACGGATGCC GGGCAATATG TGGGCCGAAC GTGGAGCCTG
5701 TGGTTCGCGG CTGTGGCATT CTTGTACGCT CCATTTTGGT TCAACCCCAT GAGTTTCGAA
5761 TGGGAGCGCG TGCGAGAGGA CGTTGAAACT TTTGTCTCGT GGATGTGCAC CACTGGGGGC
5821 TCCACGAAAA ATTCCTGGGA GTCATGGTGG AAAGAGGAAA ACGGATGGAT CAAAGCGCTG
5881 GGACCCACGG CTAAAGCGTA TCTCGTCGGT CGCTCATGTA TTTGGCTGGT TGTGGCCGCC
5941 GGATTGCTGT ATAAACCTTT GTACTTGAAT CGCAAGTTCA GCGGATTGAA CTATCTTCTG
6001 TTTCATCTAG GCATCCTCCT GGGACTTTGG CAGTTCTATC GGTTCCTGGA CAGGAGGGGC
6061 AGGACGCGGA ATCTCCCATT GCCTTATTGC TGCACGCGGC CCACGAACAT CGTTATAGGG
6121 ATGGGCATCG TCTTCCTGGT GGCTCTCATC ATCATACATT CCGAGACGAT CAAATTTTTC
6181 GTGGCTCTGT ACTACCTCGG GGCGTGGATT ACGGTGGTCC TCTCAGTTTT AGGGTTTAGA
6241 GAGCAGGCTA AGATCTTCCA TTGGATTCAT GACTGGGTCT TGGCTGTCGT CTTGATTATC
6301 CCCATCTTTC TATGCACTAT ACTTCAGTTT CCTCGGCATA TTCAAACGTG GCTGCTGTAC
6361 CACAACGCTC TTTCCCAAGG CGTGGTAATA AGCGACTTGA TTCGTCACGC GCAAAACAGC
6421 CGCGAAATGT CCAATACGGA TGATGAGCGC GCGCAGGCTC CCCQTTCACA TGCCTTGGCA
6481 TCAGCTTTAC TGAATACACC TTCATCTGTG AACCTCAGAT CAGCTTATTC ACCGGCATCA
6541 GGCGGTCCCA TGCAGATCTC TCCTGAGGAG AAAACAAGAG AGCGTCTTGT TGGCAGTGGT
6601 GGTGGCAACG GGTTTGATAC CACATCGGGC GCTTCCTGCA AACGAGAGTC ATTCAAAAGC
6661 GGACAGACAC GACCAGATCA TTCTCAGTCG ACGAGTCAGC GCCCACACCA AGATCCATCT
6721 CCAGTGTCGC CGGCAGCCTC TGAGCAATCC CCAGAGGTGT TTCAATTCCG CCAACCGACC
6781 AATTTTCCAA CACGGGAATA A
Beta glucan synthase protein
Nannochloropsis gaditana IC164 isolate
SEQ ID NO: 4
      1 MGGMLAYFWC VSWMEPNLFI SDGSAAAAAD IPQTALDVLS GIVILAGTAT GAGLMLLWRS
    61 ISIGVISGFL TLALLMLLVG GASFSGAAFT GPVVVLLACL AGVGALLLAY RGNRMSKQRM
  121 NVIVTSAFGS LVLACSYDPW GARNFLLGDL AAVSVLDWAA IGHCSLERGG CHPRVALGMW
  181 LVSTFFACLV QVYLGGDPEL RQQVLALLRR DHHPYQSLPD APTRRSDASK QEPAALPKHL
  241 RQDINKFKLA ELGVNILDLR HPVDAEEDGR SSSMDEEESK LSATLLCMFE EIQDVFGFQT
  301 HSGVNQVEHL VLLLMNQKRY EDPAYRELMP AGKGPLSDEA VDAGPVKILH DKLFKNYKRW
  361 CASLKVAPHF DTIPHSESRG TSASWNGSGL GSTGGKWFER EGDKVKMHNL LLFLLIWGEA
  421 GNLRHMPECI AWLYHTTAAC FKGSTLQTIE AVEEEYFLTH AVTPIYAVVA VDMKKSRMDH
  481 VNKKNYDDFN EFFWSRQCLA YTWTPEDMPA VQAARAKRAA GEHARPGGGE TGLIARALKG
  541 SPKTFMEKRS WLMIMLAFRR LIDFHVVTFF ILAVQGFWLN LQWDDPYYYQ LMSSVFMLMN
  601 SLGIFWATLE IWATMQDIQS PCPPFEVREE AKHGVMLRLL TRFVFLLFQA RYFGLSLEAD
  661 GLDLLPDERL SDKSVQLEAW WMYVWISVAL HSVWILDCVF QACPPLSTQV FETRNHYVKA
  721 LLDIIFPQMR TYTGKRVHEP FHKWCLYFLF WSVVITAKIC FSYQFEVSPL ALPALELADD
  781 QVNYLNKNLY LTILLIIGRW LPFVAIYLLD MIIVYSLVAG VVGFLVGLYE RLGQVCDFAG
  841 IREHFMRTPE SFYSRLIYNS EDRRPKHSRK ASHVSDLGMS RRFTSSRNNL LAAAQDDDER
  901 KPLVATNTSG MQRLGNGIRS NYNGTSTQPH YEWMNCDEAF LDIGTTKWVA FATAWNKIVE
  961 NLRETDIISN DERDMLLFYF FKGLSKSIYL PVFQTAGYVE KAARLCAEKG KEFRALPNDN
1021 VHDGDQSLKQ KRDAIKSDKQ RVDRELRELL NKDRTAYEAV AETLELTLDF LRRMLGPKHA
1081 QDVLAATFTL ESFQGSNRVM TVERAVEEGN GQGMGLILES LRLENVEKAV EALGKAVSAL
1141 KSGLPRRVIN PKRVEPVKMA IPPRERGGMV TVGSSMRRVR SKGFMSNLSL SSQDLVAVGE
1201 QAAEGAVHQS PAQPQVELDS LRDKIRDSLR IFLSTVKGII VPGAPNYLLA DVATAITNVL
1261 NGPFFWDDYY ASEELDRLAE SEAKSAVMPV LAKLQGLLTL HVGDAEPKSA EARRRLSFFV
1321 NSLFMDVPKA PSISDMMSWT VITPFYSEDV LYNRKDLEAA NEDGVNTLLY LQTLYKSDWK
1381 NFQERLGLRN DSTSWAGKAK EEIRLWASMR AQTLSRTVQG MMYYEDALHM LSVLDRDPSL
1441 MPNAESNSVQ QLIKRKFGYV VACQVYGKLK KEQDSKADDI DFLLRRFPSL RVAYIDERQS
1501 KSGESSFFSV LIPANDAGTG IEEIYRVRLP GNPVLGEGKP ENQNHAMIFS RGEHVQAIDM
1561 NQEGYFEDAY KMRNFLQEFA LTGSPDMPTT ILGFREHIFT GAVSSLANYM ALQEYSFVTL
1621 GQRVLNRPLR MRLHYGHPDL FDKLFFMQNG GISKASKGIN LSEDIFAGYN NLLRGGSVEF
1681 KEYVQVGKGR DVGMQQIYKF EAKLSQGAAE GSISRDVYPM VNRVDFFRLL TYYFGGIGHY
1741 LSSVLTVAAI WLLVYVLLSL SLFQHEKIGD RPMVPIGTLQ IVLAGVGILQ TMPLFCALLL
1801 ERGVWASLTE LAQVFISGGP LYFVFHIRTR DYYYSQTILA GGAAYKATGR GFVTQHASFA
1861 ETFRYFAASH LYLGLEMVAA LVLFACYTDA GQYVGRTWSL WFAAVAFLYA PFWFNPMSFE
1921 WERVREDVET FVSWMCTTGG STKNSWESWW KEENGWIKAL GPTAKAYLVG RSCIWLVVAA
1981 GLLYKPLYLN RKFSGLNYLL FHLGILLGLW QFYRFLDRRG RTRNLPLPYC CTRPTNIVIG
2041 MGIVFLVALI IIHSETIKFF VALYYLGAWI TVVLSVLGFR EQAKIFHWIH DWVLAVVLII
2101 PIFLCTILQF PRHIQTWLLY HNALSQGVVI SDLIRHAQNS REMSNTDDER AQAPRSHALA
2161 SALLNTPSSV NLRSAYSPAS GGPMQISPEE KTRERLVGSG GGNGFDTTSG ASCKRESFKS
2221 GQTRPDHSQS TSQRPHQDPS PVSPAASEQS PEVFQFRQPT NFPTRE*
A92 GlyS LF forward oligonucleotide
SEQ ID NO: 5
AATGCGGATGCGTTCATGGATGTG
A93 GlyS left flank (LF) forward nested oligonucletide
SEQ ID NO: 6
TCTGCGGTCCACTGACATCATCAA
A137 ok299 LF reverse olignucleotide
SEQ ID NO: 7
GAACAACGAACGCAAGCGTGTGAATCGATGCCCACAATCGAATCTCCT
A95 GlyS right flank (RF) forward oligonucleotide
SEQ ID NO: 8
GTGCCATCTTGTTCCGTCTTGCTTTGGCGTTATTCGAGCGTGAGAAGA
A96 GlyS RF reverse nested oligonucletide
SEQ ID NO: 9
AACATTAGCAAACGTAAGGCGGCG
A97 GlyS RF reverse oligonucleotide
SEQ ID NO: 10
TGCAGGGCTTACCTGTATCCAACA
BGS1 knockout construct
SEQ ID NO: 11

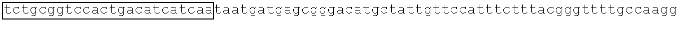

atatttatttgccagtatttcagacggcgggctcggtggagagggccgcgcggttgtgcgcggagaagggaaaagag
ttccgcaccttggctgagaaaggaaaggagctccgtgccttggccgatcaggtcgagttgcagatgcagaacgatcc
aaaccaccaccacaatcagccgtacaggaaggccatcgataacaacaaggcagagatgattaagctggatacggcat
tgtgggaggagttgtcaaaggataggacgatgcatgaagcagtggcggagacgcttgagttgagcctagaattcttg
atgcgcatgcttggggaggatcatgtatcggacgtgaataaggttaagctgacgatggagcgtctgcaggaaagcat
gaagggggacgatgcggagaagggaagggcgggggggagaaaggtgatgattttatcggggataaagctggaggaag
tggataaggctgtcggagcgttgggcaagatggtcacggcgctgaaaagtgggttgcctcgacgtgtcatcaacccg
aaccgcgtcaagcctgcaaagcacacgccgagtgcgcgggagggtcgagggacggtaacggtgggatcggcaatgaa
gaaggtgcgtagccgcgggtttatgagcaacctctccctctcctcccagaacctcgtggaagtccgggagcaggcgg
agggccaggcttccgcgtctacgcccacggccagctcgcagcctttacatgagttggacagtttgcgggataaggtg
cgggaggcactgagagggtttttgggtgcggtgaaaaagatgttagtgtctggaccgttgtttaaagatgtggcgga
agcagtggacaagattttgactggacagtttttttggtgtgatgtgtatgcatccaactccctggatcagttggcca
agcctgaggtgaaggaacttgtgcacaagatcctggcgaaactccaaggactcctcaccctgcatgtgggggatgca
gagcccaagagtgcggaggcccgtcggcggttgaccttttttgtgaattctttgttcatggatgtgccgaaggcgcc
ctctattgggaatatgttgtcatggacggtagtgacgcctttttattctgaggacgtgctctatagcagaaaggatt
tggatgcggcgaatgaggacggggtaaaaaccttactgtatctccagacgctgtataaagcggattggaaaaatttc

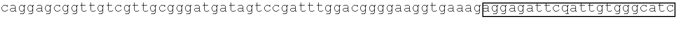

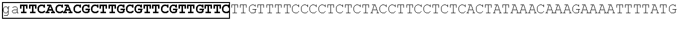


TAAAATAAGGGTGACAAAAGAAGAACCAGGGAGAAAAGAAAATGACGGGGGTAGGAAAGGACTACAGAGAAAAACAT
GATGCAGGAATTCAACACTCTCATATCAAGCAATCAGCACAAACAAACGAAGACAGCTACGGGAGAAAGGCCTTATT
TCTCTTCCGGTAGGTTAAGAAGGGATGGACAATCTCTCGCGCCAACACTGAGTGCTGCGGCTGCTACTGCTGCTGCT
ACTGCTACTACCACTGGCTCTTCCACAGAAGC
TTAGTCCTGCTCCTCGGCCACGAAGTGCACGCAGTTGCCGGCCGGGTCGCGCAGGGCGAACTCCCGCCCCCACGGCT
GCTCGCCGATCTCGGTCATGGCCGGCCCGGAGGCGTCCCGGAAGTTCGTGGACACGACCTCCGACCACTCGGCGTAC
AGCTCGTCCAGGCCGCGCACCCACACCCAGGCCAGGGTGTTGTCCGGCACCACCTGGTCCTGGACCGCGCTGATGAA
CAGGGTCACGTCGTCCCGGACCACACCGGCGAAGTCGTCCTCCACGAAGTCCCGGGAGAACCCGAGCCGGTCGGTCC
AGAACTCGACCGCTCCGGCGACGTCGCGCGCGGTGAGCACCGGAACGGCACTGGTCAACTTGGCCAT
ACTTAAGAAGTGGTGGTGGTGGTGCTGCTGCTGTAGAGGATATGGCATCGGGGGTGGGACACGAGCGGGATGTAAGT
GTTGCGATGTTTTGAGGGGTTTCGTCGGGTATGGTGCGAGTCGTGTGAAGATGTGGAGCACGTGTGGAAAAGGGCAA
GAGAACTGGGCAGAACGTATCTAGGTTTGAAAGCACTCTTCATACTTGATCGCTGGATACGCAACTCAAGGGAAAGG
TCTCTCGAAAGAACAAGAGCGAGAGCCCAGGCTCCTAGAAGGAAGAGCAAGGGGAGGTCTGTCCATGTCCAATCAGG
TAAAGCACACAAAGAGCGAAGTACAAGGTATCAGCTCTAGCAACTTGGTCAACTAGCTGGGTTTTCTTGTGACAGGG
AAAGACTGTTGAAGATAGATCAGGGGGCACTTATGGGCTCTCAAGAGGGTTGAGCTGAGCCTGTTCCCTCGCTCCGC
TTTGTCCGACGACAGAAGGCTTTGCGGGTCTTGCCCTCGGGGATCCTTACTGCAAGGTTGAGGCGTTGAGCAGACCC
CATGGGAGGTCGTTGAGGCTTTCGGCACTAAGACAAGATAGGCAAGATGCCCCAATGTCCTGTTACCAACTGGGGTG
TGGAAGCACGCCTGGAGCCTCAAGGGCTCGTTGATAAGGGGATGAAATCGTCCCGGCGAGCAAATCCTGGTTGACCT

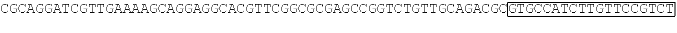

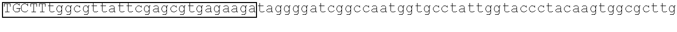


ctggtgtgggtttgctacagacagccccgctcttttgtgccttactattggagaggggaatttgggctgcgctgacg
gagctggcgcaggtgtttattagtgggggaccattgtattttgtgtttcatattcgcacacgggattacttttacac
gcaaaccattttggcgggtggtgcagcgtatagggcgacggggaggggtttcgtgacgcagcatgcttcttttgcgg
agacattccggttttttgcgttttcccacctttatttggggctggagatgattgcagccttgattttatttgcgtgt
ttcacggacgtagggcagtatgtgggtcggacgtggagtttatggtttgcggcgttggcgtttttgtacgccccttt
ttggtttaatccaatgagttttgagtgggaaagagtgagggaggacttggtgacttttgaggcttggatgcggacaa
cgggtggctcagcgtcgaactcgtgggaaacttggtggaaggaggagaataagtgggtaaaagagctgaaaaacgtc
tcggccaggctttatcttgttttgcggtcgtcgatttggttgatggtggcaacggggttgctgtataaacctatcgt
tgtggatggaaaaatggaccaattgcaatacctgctggagcacctctttgtgttgtttctgctgtttgcgacaagta
actacctggaagggagaagcaggagccgcaaccatcagggtgagtacgcgattatccgtggccttacgattatcctg
gctataattgcggttagttttttcgtcgtcacggcccagcacacggagacattcaaatttttagtggccctttacta
cattgccgcctggtgtgccacggtcatgtatgtctccaacagcaagaccgataaccttgtaaaagcctttcacaaag
cacacgactggctcctggccacttgctgcttcgtccccataggcatctgcaccataattcagttccccgcctacatc
caaacctggctcctctaccacaatgccctctctcaaggcgtcgtcatcggagatcttatccgctacgcgcagaatag
tcgggaaaccaccaatatcattgatgaacgcgccgatgcctcctcccttgcgtcaggcttgcctactcctcgttcat
ccaccatctctttgatgtccggggccaccagagctacaacagctacctctgccgctactaccgtgggaacccttcag
atctccccagaggaaaagaccaccgaacgcattgtcgaaattgagggcagcggtgggggcggatataacatactatc
ccctccgacgggtaccaagaaaaagaatggaaagaatggcacagcctcaaaagcagcgacggaattgccatggcagg
catcggttcaagatgcgcaggatccgtcggtggcagcgccgccgctgcccaatattaacactaacgcggggacggtg

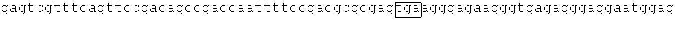

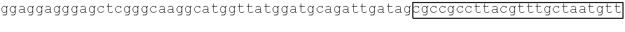


5′ arm of BGS1 KO construct
SEQ ID NO: 12
tctgcggtccactgacatcatcaataatgatgagcgggacatgctattgttccatttctttacgggttttgccaagg
atatttatttgccagtatttcagacggcgggctcggtggagagggccgcgcggttgtgcgcggagaagggaaaagag
ttccgcaccttggctgagaaaggaaaggagctccgtgccttggccgatcaggtcgagttgcagatgcagaacgatcc
aaaccaccaccacaatcagccgtacaggaaggccatcgataacaacaaggcagagatgattaagctggatacggcat
tgtgggaggagttgtcaaaggataggacgatgcatgaagcagtggcggagacgcttgagttgagcctagaattcttg
atgcgcatgcttggggaggatcatgtatcggacgtgaataaggttaagctgacgatggagcgtctgcaggaaagcat
gaagggggacgatgcggagaagggaagggcgggggggagaaaggtgatgattttatcggggataaagctggaggaag
tggataaggctgtcggagcgttgggcaagatggtcacggcgctgaaaagtgggttgcctcgacgtgtcatcaacccg
aaccgcgtcaagcctgcaaagcacacgccgagtgcgcgggagggtcgagggacggtaacggtgggatcggcaatgaa
gaaggtgcgtagccgcgggtttatgagcaacctctccctctcctcccagaacctcgtggaagtccgggagcaggcgg
agggccaggcttccgcgtctacgcccacggccagctcgcagcctttacatgagttggacagtttgcgggataaggtg
cgggaggcactgagagggtttttgggtgcggtgaaaaagatgttagtgtctggaccgttgtttaaagatgtggcgga
agcagtggacaagattttgactggacagtttttttggtgtgatgtgtatgcatccaactccctggatcagttggcca
agcctgaggtgaaggaacttgtgcacaagatcctggcgaaactccaaggactcctcaccctgcatgtgggggatgca
gagcccaagagtgcggaggcccgtcggcggttgaccttttttgtgaattctttgttcatggatgtgccgaaggcgcc
ctctattgggaatatgttgtcatggacggtagtgacgcctttttattctgaggacgtgctctatagcagaaaggatt
tggatgcggcgaatgaggacggggtaaaaaccttactgtatctccagacgctgtataaagcggattggaaaaatttc
caggagcggttgtcgttgcgggatgatagtccgatttggacggggaaggtgaaagaggagattcgattgtgggcatc
ga
3′ arm of BGS1 KO construct
SEQ ID NO: 13
tggcgttattcgagcgtgagaagataggggatcggccaatggtgcctattggtaccctacaagtggcgcttgctggt
gtgggtttgctacagacagccccgctcttttgtgccttactattggagaggggaatttgggctgcgctgacggagct
ggcgcaggtgtttattagtgggggaccattgtattttgtgtttcatattcgcacacgggattacttttacacgcaaa
ccattttggcgggtggtgcagcgtatagggcgacggggaggggtttcgtgacgcagcatgcttcttttgcggagaca
ttccggttttttgcgttttcccacctttatttggggctggagatgattgcagccttgattttatttgcgtgtttcac
ggacgtagggcagtatgtgggtcggacgtggagtttatggtttgcggcgttggcgtttttgtacgcccctttttggt
ttaatccaatgagttttgagtgggaaagagtgagggaggacttggtgacttttgaggcttggatgcggacaacgggt
ggctcagcgtcgaactcgtgggaaacttggtggaaggaggagaataagtgggtaaaagagctgaaaaacgtctcggc
caggctttatcttgttttgcggtcgtcgatttggttgatggtggcaacggggttgctgtataaacctatcgttgtgg
atggaaaaatggaccaattgcaatacctgctggagcacctctttgtgttgtttctgctgtttgcgacaagtaactac
ctggaagggagaagcaggagccgcaaccatcagggtgagtacgcgattatccgtggccttatgattatcctggctat
aattgcggttagttttttcgtcgtcacggcccagcacacggagacattcaaatttttagtggccctttactacattg
ccgcctggtgtgccacggtcatgtatgtctccaacagcaagaccgataaccttgtaaaagcctttcacaaagcacac
gactggctcctggccacttgctgcttcgtccccataggcatctgcaccataattcagttccccgcctacatccaaac
ctggctcctctaccacaatgccctctctcaaggcgtcgtcatcggagatcttatccgctacgcgcagaatagtcggg
aaaccaccaatatcattgatgaacgcgccgatgcctcctcccttgcgtcaggcttgcctactcctcgttcatccacc
atctctttgatgtccggggccaccagagctacaacagctacctctgccgctactaccgtgggaacccttcagatctc
cccagaggaaaagaccaccgaacgcattgtcgaaattgagggcagcggtgggggcggatataacatactatcccctc
cgacgggtaccaagaaaaagaatggaaagaatggcacagcctcaaaagcagcgacggaattgccatggcaggcatcg
gttcaagatgcgcaggatccgtcggtggcagcgccgccgctgcccaatattaacactaacgcggggacggtggagtc
gtttcagttccgacagccgaccaattttccgacgcgcgagtgaagggagaagggtgagagggaggaatggagggagg
agggagctcgggcaaggcatggttatggatgcagattgatagcgccgccttacgtttgctaatgtt
```

1. A modified algal cell having (1) suppressed expression or activity of endogenous beta glucan synthase 1 (BGS1); and (2) increased lipid synthesis when grown under nutrient deficient conditions.

2. The modified algal cell of claim 1, wherein the algal cell has decreased sugar content compared to a wild-type cell when grown under nutrient deficient conditions.

3. The modified algal cell of claim 2, wherein the algal cell has at least 50% less sugar content compared to a wild-type cell when grown under nutrient deficient conditions.

4. The modified algal cell of claim 1, wherein the algal cell has at least 25% more lipid content compared to a wild-type cell when grown under nutrient deficient conditions.

5. The modified algal cell of claim 1, wherein the algal cell has at least 40% lipid content by ash-free dry weight.

6. The modified algal cell of claim 1, wherein the nutrient deficient condition is nitrogen starvation.

7. The modified algal cell of claim 1, wherein suppressed expression or activity of endogenous BGS1 comprises contacting the algal cell with an inhibitor of BGS1.

8. The modified algal cell of claim 7, wherein the inhibitor of BGS1 is a siRNA, a microRNA, or an antisense RNA.

9. The modified algal cell of claim 1, wherein suppressed expression or activity of endogenous BGS1 comprises inactivating or removing the endogenous BGS1 gene by gene editing.

10.-16. (canceled)

17. A method for making the modified algal cell of claim 1, the method comprising suppressing the expression or activity of an endogenous BGS1 in an algal cell.

18. The method of claim Error! Reference source not found., wherein suppressing the expression or activity of the endogenous BGS1 comprises:
(a) transforming the algal cell with a targeting construct comprising a selectable marker,
wherein the selectable marker is flanked at the 5′ end by a first nucleic acid sequence of an endogenous BGS1 gene and at the 3′ end by a second nucleic acid sequence of the endogenous BGS1 gene, and
wherein said targeting construct integrates into the algal nuclear genome by homologous recombination, thereby inactivating or removing the BGS1 gene; and
(b) selecting the transformed algal cell carrying the inactivated BGS1 gene, thereby suppressing the expression of BGS1.

19. The method of claim Error! Reference source not found., wherein the BGS1 gene comprises the BGS1 promoter or one or more regulatory elements.

20. The method of claim Error! Reference source not found., wherein the first nucleic acid sequence of the endogenous BGS1 gene comprises about 200 bp to about 5 kb.

21. The method of claim Error! Reference source not found., wherein the second nucleic acid sequence of the endogenous BGS1 gene comprises about 200 bp to about 5 kb.

22. The method of claim Error! Reference source not found., wherein the first and second nucleic acid sequences are different lengths.

23. The method of claim Error! Reference source not found., wherein the first and second nucleic acid sequences are the same lengths.

24. The method of claim Error! Reference source not found., wherein the first and second nucleic acid sequences are non-overlapping sequences of the BGS1 gene.

25. The method of claim Error! Reference source not found., wherein the selectable marker is an antibiotic resistance gene.

26.-37. (canceled)

38. A method for obtaining at least 40% lipids by ash-free dry weight from an algal biomass derived from an algal cell grown under a nutrient deficient condition, the method comprising:
(a) cultivating any one of the algal cells of claims 1-14, under the nutrient deficient condition;
(b) generating an algal biomass from said cells; and
(c) extracting lipids from said algal biomass, wherein the lipid amount is at least about 40% lipids per ash-free dry weight.

39. The method of claim Error! Reference source not found., wherein the nutrient deficient condition is nitrogen starvation.
